(19) United States
(12) Patent Application Publication
(10) Pub. No.: US 2009/0081669 A1
(43) Pub. Date: Mar. 26, 2009

(54) Fluorescent Assays Using Orthogonal tRNA - Aminoacyl Synthetase Pairs

(75) Inventors: Nima Shiva, Los Angeles, CA (US); Mark W. Nowak, Pasadena, CA (US); Niki Zacharias, Monrovia, CA (US)

Correspondence Address: SHELDON MAK ROSE & ANDERSON PC, 100 Corson Street, Third Floor, PASADENA, CA, 91103-3842, US

(73) Assignee: ENCODE BIO, INC., Pasadena, CA (US)

(21) Appl. No.: 12/162,956

(22) PCT Filed: Feb. 1, 2007
(86) PCT No.: PCT/US07/61492

§ 371 Date: Jul. 31, 2008

Related U.S. Application Data
(60) Provisional application No. 60/764,470, filed on Feb. 1, 2006.
(60) Provisional application No. 60/864,389, filed on Nov. 3, 2006.

(51) Int. Cl. C12Q 1/68

(52) U.S. Cl. 435/6

(57) ABSTRACT

An unnatural amino acid comprising a fluorescent moiety is site-specifically incorporated into a protein through the use of an O-tRNA/O-RS pair which is orthogonal to the tRNAs and aminoacyl synthetases of a translation system, thereby allowing the protein to be analyzed.

### BACKGROUND

Protein structure and function have a direct impact on human health. Protein aggregation and misfolding, for example, have been implicated in a number of disease states, including Alzheimer's disease and CJD (Creutzfeldt-Jakob disease). The study of protein structure and function is therefore an important aspect of medical research.

Ideally, the study of protein structure and function should not involve steps that might perturb the structure of a protein under evaluation. Current techniques for site-specifically labeling proteins, however, often involve manipulations that can affect protein structure. For example, cysteine labeling of proteins with a thiol-fluorophore reagent often requires extensive mutagenesis of the target protein in order to obtain a reactive cysteine moiety with which a reagent can react. By potentially introducing changes to the structure and/or function of a protein being studied, the results of an assay making use of the protein can be called into question.

### SUMMARY

The present compositions, systems, and methods allow a fluorescent moiety to be site-specifically incorporated into a protein without introducing extensive changes into the protein molecule, thereby allowing protein structure and function at a particular position along a polypeptide chain to be reliably studied. The present methods can be accomplished either in vitro or in vivo using a variety of translation systems, in particular eukaryotic translation systems. In one aspect, the present methods comprise an assay to determine a property of a protein by providing a translation system comprising tRNAs and aminoacyl synthetases; providing an O-tRNA/O-RS pair which is orthogonal to the tRNAs and aminoacyl synthetases of the translation system, the O-tRNA is aminoacylated by the O-RS with a label, and the label is an unnatural amino acid molecule comprising a fluorescent moiety or a reactive unnatural amino acid molecule; providing an mRNA molecule coding for the protein, the mRNA molecule comprises a selector codon; translating the mRNA molecule with the translation system and the O-tRNA/O-RS pair, the O-tRNA comprises an anticodon loop that specifically binds the selector codon of the mRNA molecule, thereby site-selectively incorporating the label into the protein; exciting the fluorescent moiety of label; and measuring an emitted optical signal produced in response to the excitation of the fluorescent moiety, thereby determining the property of the protein. If the label comprises a reactive unnatural amino acid molecule, the method can further include providing a fluorescent molecule comprising a reactive moiety as well as a fluorescent moiety, and then reacting the reactive unnatural amino acid with the reactive moiety of the fluorescent molecule, thereby attaching the fluorescent moiety to the label.

The fluorescent moiety of the label is preferably a polarity-sensitive fluorophore, and is preferably incorporated into the protein so as to be exposed to a hydrophobic environment when the protein is in a first conformational state and to a hydrophilic environment when the protein is in a second conformational state. In this embodiment the property determined in step (f) is the conformational state of the protein, and the method can further comprise the steps of contacting the protein with a target molecule and determining the conformational state of the protein in the presence of the target molecule. If the protein is a kinase the target molecule preferably binds outside of the kinase's ATP-binding site. The protein is preferably an enzyme, such as an ATPase, a lipase, a phosphatase, a phosphodiesterase, or a kinase.

The translation system used in the present methods, which can be an in vitro system, preferably comprises components of a eukaryotic cell, such as a member of the Animalia and Fungi kingdoms. Examples include components of a yeast cell or insect cell, though such components can also be those belonging to other members of the Mammalia and Amphibia groups. If the translation system is a eukaryotic translation system, the O-RS/O-tRNA pair is preferably derived from a prokaryote, such as L. lactis. If the translation system is an in vivo system, the fluorescent moiety of the unnatural amino acid is excited and detected while it is in the cell.

In the present methods, the selector codon can be selected from the group consisting of an amber codon, an opal codon, an ocher codon, and a four base codon, and the O-RS is preferably derived from a tyrosyl aminoacyl synthetase. With respect to the unnatural amino acids used in the present methods, they can be a tyrosine analog, a glutamine analog, a phenylalanine analog, serine analog, a threonine analog, a β-amino acid, and a cyclic amino acid other than proline. For example, an unnatural amino acid can be a derivative of a natural amino acid comprising a substitution or addition selected from the group consisting of an alkyl group, an aryl group, an acyl group, an azido group, a cyano group, a halo group, a hydrazine group, a hydrazide group, a hydroxyl group, an alkenyl group, an alkynl group, an ether group, a thiol group, a sulfonyl group, a seleno group, an ester group, a thioacid group, a borate group, a boronate group, a phospho group, a phosphono group, a phosphine group, a heterocyclic group, an enone group, an imine group, an aldehyde group, a hydroxylamino group, a keto group, a sugar group, α-hydroxy group, a cyclopropyl group, a cyclobutyl group, a cyclopentyl group, a 2-nitrobenzyl group, a 3,5-dimethoxy-2-nitrobenzyl group, a 3,5-dimethoxy-2-nitroveratrole carbamate group, a nitrobenzyl group, a 3,5-dimethoxy-2-nitrobenzyl group, and an amino group.

In one embodiment, the unnatural amino acid used in the present methods is a reactive unnatural amino acid, such as a halogenated phenyalanine derivative, an unnatural amino acid containing an azide moiety, an unnatural amino acid containing an acetylene moiety, or an unnatural amino acid containing an acetyl group. For example, such an unnatural amino acid can be 2-F-phenylalanine, 3-F-phenylalanine, 4-F-phenylalanine, 2-Br-phenylalanine, 3-Br-phenylalanine, 4-Br-phenylalanine, 2-Cl-phenylalanine, 3-Cl-phenylalanine, 4-Cl-phenylalanine, 4-CN-phenylalanine, p-azido-phenylalanine, o-azido-phenylalanine, 2-amino-2-(4-(ethynyloxy)phenyl)acetic acid, p-acetyl-phenylalanine, p-ethynyl-phenylalanine, 2-(4-allylphenyl)-2-aminoacetic acid, 2-amino-4-oxopentanoic acid, or 2-amino-5-oxohexanoic acid.

The fluorescent moities used in the present methods can be a dansyl group, an anthraniloyl group, an acrylodan group, a coumarin group, a 4-nitrobenzo[c][1,2,5]oxadiazole (NBD) group, and a dipyrrometheneboron difluoride (BODIPY) group. For example, such a fluorescent moiety can be 4-nitrobenzo[c][1,2,5]oxadiazole (NBD), acrylodan, dansylalanine, dansylysine, dansyl-dap, 7-azatryptophan, 3-anthraniloyl-2-amino propionic acid (AtnDap), 6-dimethylamino-2-acyl-napthalene alanine, (ALADAN), α-amino-3-[6,7dimethoxy-2-oxo-2H-chromen-4-ylmethyl)-amino]-propionic acid, 2-amino-3-(7-nitro-benzo[1,2,5]oxadiazol-4-ylamino)propionic acid (NBD-Dap), 2-amino-3-BODIPY-propionic acid, 2-amino-6-BODIPY-hexanoic acid, 2-hydroxy-3-BODIY-propionic acid, or 2-hydroxy-6-BODIPY-hexanoic acid. If the fluorescent moiety is one to be reacted with a reactive unnatural amino acid, it can comprise a reactive group such as an alcohol moiety, a hydrazide moiety, an ethene moiety, an acetylene moiety, or an azide moiety.

The present methods can be used, for example, to evaluate the dimerization of a protein, in which case the method can involve exciting a fluorescent moiety with polarized light. Protein aggregation can also be studied with the present methods. In addition, in some embodiments of the present methods, two labels can be incorporated into a protein at positions which allow a FRET interaction between the two labels to occur.

The present systems include the foregoing elements for accomplishing the present methods, and can be either cell-free (in vitro) or contained in a cell. Cells comprising such components are also included herein.

### DRAWINGS

These and other features, aspects and advantages of the present invention will become better understood with regard to the following description, appended claims, and accompanying figures where:

FIGS. 1A-1D illustrate the incorporation of an unnatural amino acid into a protein using an O-RS/O-tRNA pair.

FIG. 2 illustrates the difference in fluorescent signal strength between Abelson kinase in an active conformation and in an inactive conformation.

FIG. 3A depicts plasmid ptRNA_{CUA}/ADH1-TyrRS.

FIG. 3B depicts plasmid pYeastSelection (GAL4).

All dimensions specified in this disclosure are by way of example only and are not intended to be limiting. Further, the proportions shown in these Figures are not necessarily to scale. As will be understood by those with skill in the art with reference to this disclosure, the actual dimensions of any device or part of a device disclosed in this disclosure will be determined by their intended use.

### DESCRIPTION

The present compositions, systems, and methods allow proteins to be labeled with fluorescent moieties in a site-specific manner without substantially altering the structure of a protein, and thereby allow proteins labeled in this way to be reliably assayed. While any number of proteins can be labeled using the present methods, these methods are believed to be particularly useful in studying enzymes, which play important roles in a variety of cellular functions. Other applications of the present technology will become apparent in view of the following description and examples.

### DEFINITIONS

As used herein, the following terms and variations thereof have the meanings given below, unless a different meaning is clearly intended by the context in which such term is used.

“About” and “approximately” generally means an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” can mean values that are within an order of magnitude, preferably within 5-fold and more preferably within 2-fold of a given value. Numerical quantities given herein are approximate unless stated otherwise, meaning that the term “about” or approximately can be inferred when not expressly stated.

“Analog” means a molecule which resembles another molecule in structure, such as a molecule which comprises a portion of the chemical structure or polymer sequence of another molecule, but which is not identical to or an isomer of such other molecule.

“Derived from” and “derivative” refer to a composition or component which is: (1) isolated from a source, such as from a particular organism; (2) isolated from a source and then modified; or (3) formed from a particular molecule or starting material, i.e. a modified form of such starting molecule or material. Also included are compositions and components that are generated (e.g., chemically synthesized or recombinantly produced) using sequence, chemical composition, structure, or other information about such a derived composition or component.

“Eukaryote” and “eukaryotic” refer to organisms belonging to the phylogenetic domain Eucarya, including those belonging to the taxonomic kingdoms Animalia and Fungi, such as animals (e.g., mammals, insects, reptiles, and birds) and fungi (such as yeasts).

“Fluorescent unnatural amino acid” means an unnatural amino acid (defined below) which includes a fluorophore. Fluorescent unnatural amino acids include natural amino acids or derivatives thereof to which a fluorescent moiety is bound.

“Kinase” means an enzyme that catalyzes the transfer of a phosphate group from a donor, such as ADP or ATP, to an acceptor molecule. Kinases in biological systems phosphorylate proteins, DNA, saccharides, and lipids. “Protein kinase” means a kinase that catalyzes the transfer of a phosphate group from a donor molecule to an acceptor protein or peptide molecule.

“Natural amino acid” means selenocysteine and the following twenty alpha-amino acids: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine.

“Negative selection marker” refers to a detectable indicator than, when present, e.g., expressed in a cell, activated or the like, allows identification of an organism that does not possess a particular property (e.g., as compared to an organism which does possess the property). A “positive selection marker” conversely refers to an indicator than when present, e.g., expressed, activated or the like, results in the ability to identify an organism with the positive selection marker and distinguish it from organisms which lack the positive selection marker.

“Orthogonal” refers either to a tRNA molecule or to an aminoacyl synthetase molecule which reacts with reduced efficiency with the endogenous components of a translation system, i.e. with components derived from a particular organism or organisms. Reduced efficiency refers to a lesser ability of an orthogonal component to aminoacylate or be aminoacylated by an endogenous component of a cell or other translation system, and can be, e.g., to a level of less than 20% as efficient as an endogenous component, less than 10% as efficient, less than 5% as efficient, or less than 1% as efficient, with efficiency being measured by K_{cat}/Kₘ. For example, an orthogonal tRNA in a translation system of interest is aminoacylated by any endogenous aminoacyl synthetase of the translation system with reduced or even zero efficiency, when compared to aminoacylation of an endogenous tRNA by the endogenous aminoacyl synthetase of the translation system. In another example, an orthogonal aminoacyl synthetase aminoacylates any endogenous tRNA in the translation system of interest with reduced or even zero efficiency as compared to aminoacylation of the endogenous tRNA by an endogenous aminoacyl synthetase.

“O-RS” means an orthogonal aminoacyl-tRNA synthetase. “RS” refers to an aminoacyl-tRNA synthetase (i.e., aminoacyl synthetase).

“O-tRNA” means orthogonal tRNA.

“Preferential aminoacylation” means aminoacylation of a tRNA molecule with greater efficiency, i.e. with a higher K_{cat}/Kₘ. Preferential aminoacylation is preferably at an efficiency of greater than about 70% efficient, and more preferably of greater than about 80% efficient. In preferred embodiments, preferential aminoacylation occurs at an efficiency of greater than about 90%, such as at an efficiency of about 95%-99% or higher. With respect to an O-RS, preferential aminoacylation generally refers to the aminoacylation of O-tRNA with an unnatural amino acid (in particular one comprising a reactive group or a fluorescent moiety) at greater efficiency compared to aminoacylation of a naturally occurring tRNA with the amino acid.

“Reactive unnatural amino acid” means an unnatural amino acid (defined below) which can be reacted with and thereby joined to a fluorescent dye post-translationally.

“Reporter” means a measurable composition or characteristic of a composition, or another component of a system which codes for or results in the production of such a composition, such as a green fluorescent protein, β-galactosidase, or a nucleic acid which encodes such a protein.

“Selector codon” means a codon (i.e., a series of 3 or more nucleic acids) recognized by an O-tRNA in the translation process and not recognized by an endogenous tRNA. The O-tRNA anticodon loop recognizes the selector codon on an mRNA so that the amino acid it carries, e.g. an unnatural amino acid, is incorporated at the site in the polypeptide encoded by the selector codon.

“Translation system” refers to the biochemical components, e.g. of a cell, necessary to incorporate an amino acid into a growing polypeptide chain (a peptide or protein). Such components include, e.g., ribosomes, tRNAs, synthetases, and mRNA. The components of a translation system can be present either in vitro or in vivo.

“Unnatural amino acid” means any amino acid, amino acid derivative, amino acid analog, α-hydroxy acid, or other molecule, other than a natural amino acid, which can be incorporated into a polypeptide chain with an O-tRNA/O-RS pair and which allows extension of the polypeptide chain. Unnatural amino acids include both reactive unnatural amino acids and fluorescent unnatural amino acids.

As used herein, the term “comprise” and variations of the term, such as “comprising” and “comprises,” are not intended to exclude other additives, components, integers or steps. The terms “a,” “an,” and “the” and similar referents used herein are to be construed to cover both the singular and the plural unless their usage in context indicates otherwise.

Orthogonal tRNAs and Orthogonal Aminoacyl-tRNA Synthetases

The proteins used in the present methods are produced using a translation system, either in vitro or in vivo, together with O-RS/O-tRNA pairs that incorporate unnatural amino acids into such proteins. The translation system preferably comprises translation components of a eukaryotic organism, and the O-RS/O-tRNA pair is preferably derived from a prokaryotic organism such as L. lactis, G. oxydans, R. rubrum or E. coli. Preferred O-RS/O-tRNA pairs include TyrRS/tRNA pairs from L. lactis, G. oxydans, and R. rubrum and LeuRS/tRNA pairs derived from E. coli, which are orthogonal to eukaryotic TyrRS/tRNA pairs. The O-tRNAs are selected so as to recognize a selector codon, such as an amber stop codon, placed in frame at any position in an mRNA molecule coding for a protein of interest.

An orthogonal tRNA for use in the present systems and methods recognizes a selector codon and is preferentially aminoacylated in a translation system with an unnatural amino acid comprising a reactive group or a fluorescent moiety by an orthogonal aminoacyl-tRNA synthetase. The O-tRNA is not preferentially acylated by endogenous synthetases. The O-tRNA can be, e.g., a suppressor tRNA. In one embodiment, the O-tRNA is encoded by a nucleotide sequence selected from one of SEQ ID NOS. 1-4, which encode a tRNA molecules derived from L. lactis tyrosyl tRNA. Alternatively, the O-tRNA is encoded by a nucleotide sequence selected from one of SEQ ID NOS. 5-7, which encode tRNA molecules derived from E. coli LeutRNA.

In order to specifically incorporate an unnatural amino acid into a protein with a translation system, either in vitro or in vivo, the substrate specificity of an orthogonal aminoacyl-tRNA synthetase (such as L. lactis tyrosyl tRNA synthetase, whose nucleotide/amino acid sequences are SEQ ID NOS:8-9) is altered so that only the desired unnatural amino acid, but not any of the common 20 amino acids, are charged to the corresponding O-tRNA. The efficiency of incorporation of an unnatural amino acid into a protein as compared to the incorporation of a natural amino acid can be, e.g., greater than about 75%, greater than about 85%, greater than about 95%, or greater than about 99%. Preferably, the orthogonal aminoacyl-tRNA synthetases have improved or enhanced enzymatic properties, e.g., the Kₘ is lower, the k_{cat} is higher, and/or the value of k_{cat}/Kₘ is higher, for the unnatural amino acid as compared to a natural amino acid.

### Selector Codons

Selector codons in mRNA molecules allow unnatural amino acids comprising a reactive group or a fluorescent moiety to be incorporated into proteins using O-RS/O-tRNA pairs. Selector codons can comprise a nonsense codon (i.e., a codon not recognized by the translation system as coding for a natural amino acid), an unnatural codon (i.e., a codon comprising an unnatural nucleic acid base pair), a stop codon such as an amber (TAG/UAG), ochre (TAA/UAA), or opal (TGA/UGA) codon, or a four (or more) base codon, e.g., AGGA, CUAG, UAGA, or CCCU. For a given system a selector codon can also include one of the natural three base codons, if the translation system does not use the natural three base codon, i.e. is a system lacking a tRNA that recognizes the natural three base codon.

A number of selector codons can be introduced into a desired nucleic acid sequence, e.g., one or more, two or more, or more than three. As a result, a number of labeled unnatural amino acids (the same and/or different unnatural amino acids) can be incorporated precisely into the polypeptide chain of a protein.

### Unnatural Amino Acids

The unnatural amino acids used in the present methods and systems can be any of a variety of amino acids, amino acid derivatives, amino acid analogs, α-hydroxy acids, or other molecules which can be incorporated into an amino acid chain in substitution for a natural amino acid. Unnatural amino acids are used to label a polypeptide in the present methods, and therefore include either a fluorescent moiety or a reactive moiety with which a fluorescent moiety can be reacted in order to attach the fluorescent moiety to the unnatural amino acid. Such unnatural amino acids preferably do not significantly alter the conformation of the peptides or proteins into which they are incorporated.

The unnatural amino acids of the present systems and methods generally have a carboxylic acid and an amine or hydroxy acid on the alpha carbon of the molecule in order to allow them to be incorporated into a polypeptide chain and allow for extension of the polypeptide chain. Preferably, an unnatural amino acid comprises one of the following structures:

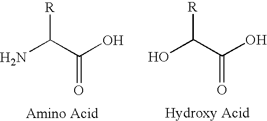

The R group should include either a fluorophore or reactive group that can be reacted with a fluorescent dye in order to attach a fluorescent moiety to the R group. The R group can be, for example, an alkyl group, an aryl group, an acyl group, an azido group, a cyano group, a halo group, a hydrazine group, a hydrazide group, a hydroxyl group, an alkenyl group, an alkynl group, an ether group, a thiol group, a sulfonyl group, a seleno group, an ester group, a thioacid group, a borate group, a boronate group, a phospho group, a phosphono group, a phosphine group, a heterocyclic group, an enone group, an imine group, an aldehyde group, a hydroxylamino group, a keto group, a sugar group, α-hydroxy group, a cyclopropyl group, a cyclobutyl group, a cyclopentyl group, a 2-nitrobenzyl group, a 3,5-dimethoxy-2-nitrobenzyl group, a 3,5-dimethoxy-2-nitroveratrole carbamate group, a nitrobenzyl group, a 3,5-dimethoxy-2-nitrobenzyl group, or an amino group. R can also be a fluorescent moiety like a dansyl group, an anthraniloyl group, an acrylodan group, a coumarin group, an 4-nitrobenzo[c][1,2,5]oxadiazole (NBD) group, and a dipyrrometheneboron difluoride (BODIPY) group. Fluorophores can be attached to or otherwise included in the amino acids (unnatural amino acids) used in the present systems and methods in ways known to the art.

An unnatural amino acid in the present systems and methods can be derived from natural amino acids and can be, for example, a tyrosine analog, a glutamine analog, a phenylalanine analog, serine analog, a threonine analog, a α-amino acid, a β-amino acid, or a cyclic amino acid other than proline. In particular, the unnatural amino acid can comprise an analog or derivative of any of the following compounds: hydroxy methionine, norvaline, O-methylserine. crotylglycine, hydroxy leucine, allo-isoleucine, norleucine, α-aminobutyric acid, t-butylalanine, hydroxy glycine, hydroxy serine, F-alanine, hydroxy tyrosine, homotyrosine, 2-F-tyrosine, 3-F-tyrosine, 4-methyl-phenylalanine, 4-methoxy-phenylalanine, 3-hydroxy-phenylalanine, 4-NH₂-phenylalanine, 3-methoxy-phenylalanine, 2-F-phenylalanine, 3-F-phenylalanine, 4-F-phenylalanine, 2-Br-phenylalanine, 3-Br-phenylalanine, 4-Br-phenylalanine, 2-Cl-phenylalanine, 3-Cl-phenylalanine, 4-Cl-phenylalanine, 4-CN-phenylalanine, 2,3-F₂-phenylalanine, 2,4-F₂-phenylalanine, 2,5-F₂-phenylalanine, 2,6-F₂-phenylalanine, 3,4-F₂-phenylalanine, 3,5-F₂-phenylalanine, 2,3-Br₂-phenylalanine, 2,4-Br₂-phenylalanine, 2,5-Br₂-phenylalanine, 2,6-Br₂-phenylalanine, 3,4-Br₂-phenylalanine, 3,5-Br₂-phenylalanine, 2,3-Cl₂-phenylalanine, 2,4-Cl₂-phenylalanine, 2,5-Cl₂-phenylalanine, 2,6-Cl₂-phenylalanine, 3,4-Cl₂-phenylalanine, 2,3,4-F₃-phenylalanine, 2,3,5-F₃-phenylalanine, 2,3,6-F₃-phenylalanine, 2,4,6-F₃-phenylalanine, 3,4,5-F₃-phenylalanine, 2,3,4-Br₃-phenylalanine, 2,3,5-Br₃-phenylalanine, 2,3,6-Br₃-phenylalanine, 2,4,6-Br₃-phenylalanine, 3,4,5-Br₃-phenylalanine, 2,3,4-Cl₃-phenylalanine, 2,3,5-Cl₃-phenylalanine, 2,3,6-Cl₃-phenylalanine, 2,4,6-Cl₃-phenylalanine, 3,4,5-Cl₃-phenylalanine, 2,3,4,5-F₄-phenylalanine, 2,3,4,5-Br₄-phenylalanine, 2,3,4,5-Cl₄-phenylalanine, 2,3,4,5,6-F₅-phenylalanine, 2,3,4,5,6-Br₅-phenylalanine, 2,3,4,5,6-Cl₅-phenylalanine, cyclohexylalanine, hexahydrotyrosine, cyclohexanol-alanine, hydroxyl alanine, hydroxy phenylalanine, hydroxy valine, hydroxy isoleucine hydroxyl glutamine, thienylalanine, pyrrole alanine, NT-methyl-histidine, 2-amino-5-oxohexanoic acid, norvaline, norleucine, 3,5-F₂-phenyalanine, cyclohexyalanine, 4-Cl-phenyalanine, p-azido-phenylalanine, o-azido-phenylalanine, 2-(4-allylphenyl)-2-aminoacetic acid, O-4-allyl-L-tyrosine, 2-amino-4-pentanoic acid, and 2-amino-5-oxohexanoic acid. Such analogs and derivatives further comprise a fluorescent or reactive moiety in the present methods.

When the unnatural amino acid of the present methds and systems is a reactive unnatural amino acid, it can be, for example, a halogenated phenyalanine derivative, an unnatural amino acid containing an azide moiety, an unnatural amino acid containing an acetylene moiety, an unnatural amino acid containing an ethene moiety, or an unnatural amino acid containing an acetyl group. Reactive unnatural amino acids that include acetyl groups can be coupled to fluorescent moieties containing a hydrazide. Unnatural amino acids containing azide or acetylene moieties can be coupled to fluorescent moieties using “click” chemistry (e.g., involving a 3+2 cycloaddition reaction). Examples of reactive unnatural amino acids include 2-F-phenylalanine, 3-F-phenylalanine, 4-F-phenylalanine, 2-Br-phenylalanine, 3-Br-phenylalanine, 4-Br-phenylalanine, 2-Cl-phenylalanine, 3-Cl-phenylalanine, 4-Cl-phenylalanine, 4-CN-phenylalanine, p-azido-phenylalanine, o-azido-phenylalanine, 2-amino-2-(4-(ethynyloxy)phenyl)acetic acid, p-acetyl-phenylalanine, p-ethynyl-phenylalanine, 2-amino-4-oxopentanoic acid, and 2-amino-5-oxohexanoic acid.

Fluorescent moieties that can be reacted with a reactive moiety of a reactive unnatural amino acid include dansyl groups, anthraniloyl groups, acrylodan groups, coumarin groups, 4-nitrobenzo[c][1,2,5]oxadiazole (NBD) groups, fluorescein groups, and dipyrrometheneboron difluoride (BODIPY) groups, as long as such fluorescent moieties comprise a reactive moiety, which can preferably be an alcohol moiety, a hydrazide moiety, an ethene moiety, an acetylene moiety, or an azide moiety. The reactive moiety of such fluorophores is preferably coupled to the reactive moiety of a reactive unnatural amino acid after the reactive unnatural amino acid has been incorporated into a protein by methods known to those of skill in the art.

### Fluorophores

A number of fluorescent moieties can be used in the present methods and systems. Fluorescent compounds which can be used as the fluorescent moiety include such dyes as fluorescein, rhodamine, coumarin, and derivatives thereof. Further fluorescent dyes are listed in Table 1 below, all of which are commercially available (e.g., from Sigma Chemical, St. Louis, Mo.).

TABLE 1
Fluorescent Moieties

| | |
|---|---|
| 4-acetamido-4′- | 5-(4,6-dichlorotriazin-2-yl) |
| isothiocyanatostilbene- | aminofluorescein (DTAF) |
| 2-2′-disulfonic acid | |
| 7-amino-4-trifluoromethylcoumarin | N-(4-anilino-1-naphthyl) maleimide |
| 4′,6-diamidino-2-phenylindole | 7-amino-4-methylcoumarin (AMC) |
| (DAPI) | |
| 4,4′-dilsothiocyanatostilbene-2,2′- | tetramethylrhodamine isothiocyanate |
| disulfonic acid | (TRITC) |
| quinolizino fluorescein | dansyl chloride |
| isothiocyanate (QFITC) | |
| eosin isothiocyanate | erythrosin B |
| fluorescamine | fluorescene |
| fluorescein derivatives | 4-methylumbelliferone |
| o-phthaldialdehyde | rhodamine B and derivatives |
| rhodamine 6G | rhodamine 123 |
| sulforhodamine B | sulforhodamine 101 |
| sulforhodamine 101 acid chloride | |

The unnatural amino acids used in the present methods and systems preferably comprise unnatural amino acids containing dansyl like dansylysine; tryptophan analogs like 7-azatryptophan; anthraniloyl containing unnatural amino acids like 3-anthraniloyl-2-amino propionic acid (AtnDap); acrylodan containing unnatural amino acids like 6-dimethylamino-2-acyl-napthalene alanine (ALADAN); coumarin containing unnatural amino acids like 2-amino-3-[6,7dimethoxy-2-oxo-2H-chromen-4-ylmethyl)-amino]-propionic acid; NBD containing unnatural amino acids like 2-amino-3-(7-nitro-benzo[1,2,5]oxadiazol-4-ylamino)propionic acid (NBD-Dap); and dipyrrometheneboron difluoride (BODIPY) containing unnatural amino acids like 2-amino-3-BODIPY-propionic acid or 2-amino-6-BODIPY-hexanoic acid. In addition, the hydroxy acid version of any of these fluorescent unnatural amino acids can be used, such as 2-hydroxy-3-BODIY-propionic acid and 2-hydroxy-6-BODIPY-hexanoic acid. When BODIPY analogs are used, the BODIPY side chain can be tethered to an unnatural amino acid in a number of different ways, for example via an amide linkage, a sulfur bond, or a carbon-carbon bond.

Particularly preferred in the present systems and methods are fluorescent moieties which are sensitive to the polarity of the environment to which they are exposed, i.e. fluorescent moieties whose fluorescence intensity changes depending on the polarity (hydrophilicity or hydrophobicity) of the fluorophore's environment. Such polarity-sensitive fluorophores include the following:

nitrobenzoxadiazole (NBD):

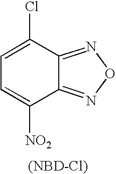

6-acryloyl-2-dimethylaminonaphthalene (acrylodan):

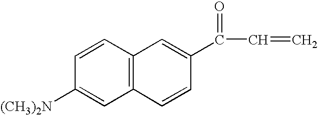

dansyl fluorophores:

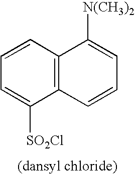

and some coumarin dyes.

NBD, for example, displays minimum fluorescence intensity in aqueous media but undergoes a large increase in fluorescence intensity when exposed to a more hydrophobic environment. When a fluorophore-labeled amino acid of this nature is incorporated into a protein of interest at a site exposed to an aqueous environment, if it is subsequently sequestered inside the protein or surrounded by another protein (i.e. “buried” in a more hydrophobic environment), it will display a detectable increase in fluorescence signal. Amino or α-hydroxy acids, for example, can be labeled with such fluorophores using methods known to those of skill in the art to produce the unnatural amino acids of the present methods.

Other polarity-sensitive fluorophores for use in the present methods include, for example, dansylysine:

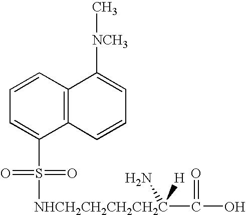

and dansylalanine:

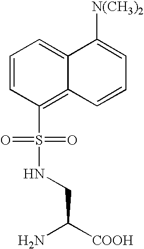

An especially preferred fluorescent unnatural amino acid is 2-amino-3-(7-nitro-benzo[1,2,5]oxadiazol-4-ylamino)propionic acid (NBD-DAP). The structure of NBD-DAP is as follows:

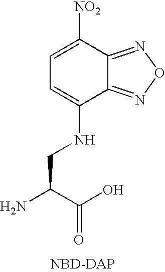

The small size of this molecule allows it to be incorporated into a protein with minimal perturbation of the structure of the protein, as compared to a protein having a natural amino acid in place of the NBD-DAP molecule.

Other fluorescent compounds which can be used include nanocrystals, also referred to as quantum dots, such as CdSe, ZnS, and PbSe. In addition, in embodiments in which fluorescence resonance energy transfer (also referred to as Förster resonance energy transfer or FRET) is employed, quenchers such as DABCYL, BHQ, or QSY dye (available from Molecular Probes, Eugene, Oreg.) can be used as FRET acceptors in some embodiments.

### Translation Systems

The translation systems with which an O-RS/O-tRNA pair is used in the present systems and methods are preferably derived from eukaryotic organisms, in particular those belonging to the taxonomic kingdoms Animalia and Fungi, such as animals (e.g., mammals, insects, reptiles, and birds) and fungi (such as yeast). Such translation systems are preferred when the enzyme or other protein into which an unnatural amino acid is being incorporated is that of a eukaryotic organism. Particularly preferred cells for use in the present method include insect cell expression systems (e.g., the Sf9 cell line, available from Orbigen, Inc., San Diego, Calif.), with which baculovirus vectors can be used, as well as those of eukaryotes from the taxonomic classes Mammalia and Amphibia, such as human cells (e.g., HEK cells), CHO cells, BHK cells and Xenopus oocytes.

Prokaryotic translation systems can also be used in some embodiments. In this case, the O-RS/O-tRNA pair can be derived from organisms such as Methanococcus jannaschii, Methanobacterium thernoautotrophicum, Halobacterium, E. coli, A. fulgidus, Halobacterium, P. furiosus, P. horikoshii, A. pernix, and T. thermophilus.

Both in vitro and in vivo translation systems can be used in the present methods. When the present methods are conducted in host cells in vivo, such host cells generally are genetically engineered (e.g., transformed, transduced or transfected) with vectors in order to provide O-RS and/or O-tRNA molecules in such cells. The vector can be, for example, a cloning vector or an expression vector, and can be in the form of plasmid (e.g., pcDNA3.1), a bacterium, a virus, a naked polynucleotide, or a conjugated polynucleotide. The vectors are introduced into cells and/or microorganisms by standard methods including electroporation, infection by viral vectors, or high velocity ballistic penetration by small particles with the nucleic acids [Klein et al., Nature 327, 70-73 (1987)]. Unnatural amino acids comprising fluorescent moieties can then be taken up by or otherwise transported into a cell. If an in vitro translation system is used in the present methods, the translation components can be produced by homologous recombination, such as through the use of an insect line and a baculovirus vector, or can be isolated from cells.

### Methods of Producing O-RS/O-tRNA Pairs

One strategy for generating an orthogonal tRNA/synthetase pair involves transforming a host cell, such as a mammalian cell, with a tRNA/synthetase pair from another organism, e.g., L. lactis. The properties of the heterologous synthetase candidate include, e.g., that it does not preferentially charge any host cell tRNA, and the properties of the heterologous tRNA candidate include that it is not preferentially acylated by any host cell synthetase. Using this approach, an O-RS can be produced by generating a pool of mutant synthetases from the framework of a wild-type synthetase from one organism, transforming host cells with vectors carrying such mutant synthetases as well as wild-type O-tRNA charged by the O-RS, and then selecting for mutated RS molecules based on their specificity for charging the O-tRNA with an unnatural amino acid relative to natural amino acids. The O-tRNA charged by this O-RS carries an anticodon loop that recognizes a selector codon, or the anticodon is otherwise mutated to recognize such a selector codon.

An orthogonal aminoacyl synthetase can be produced, for example, by mutating the synthetase, e.g., at the active site in the synthetase, at the editing mechanism site in the synthetase, and/or at different sites by combining different domains of synthetases, and applying a selection process. Preferably, a library of mutant RS molecules is generated. Such a library can be generated using various mutagenesis techniques known in the art, for example by generating site-specific mutations, random point mutations, chimeric constructs, or by employing in vivo homologous recombination. Optionally, more mutations can be introduced into an O-RS candidate by further mutagenesis to generate a second-generation synthetase library, which is used for further rounds of selection until a mutant synthetase with desired activity is evolved.

In one embodiment, an in vivo selection/screening strategy is used which is based on the combination of a positive selection step followed by a negative selection step. In the positive selection step, suppression of the selector codon introduced at a nonessential position or positions of a positive marker allows cells to survive under positive selection pressure. In the presence of both natural and unnatural amino acids, survivors thus encode active synthetases charging the orthogonal suppressor tRNA with either a natural or unnatural amino acid. In the negative selection step, suppression of a selector codon introduced at a nonessential position or positions of a negative marker removes synthetases with natural amino acid specificities. Survivors of the negative and positive selection steps encode synthetases that aminoacylate (charge) the orthogonal tRNA with unnatural amino acids only. These synthetases can then be subjected to further mutagenesis, e.g., DNA shuffling or other recursive mutagenesis methods, for example to allow them to be expressed efficiently in a host cell. These steps can be carried out in different orders in order to identify O-RS/O-tRNA pairs, such as by employing a negative selection/screening followed by positive selection/screening or further combinations thereof.

For example, a selector codon, e.g., an amber codon, can be placed in a reporter gene, e.g., an antibiotic resistance gene such as β-lactamase (which confers ampicillin resistance), with a selector codon, e.g., TAG. This construct is placed in an expression vector with members of a mutated O-RS library. This expression vector along with an expression vector with an orthogonal tRNA, e.g., a orthogonal suppressor tRNA, are introduced into a cell, which is grown in the presence of a selection agent, e.g., antibiotic media, such as ampicillin. Only if the synthetase is capable of aminoacylating (charging) the suppressor tRNA with some amino acid does the selector codon get decoded, allowing survival of the cell on antibiotic media. In the negative selection step, those synthetases with specificities for natural amino acids charge the orthogonal tRNA, resulting in suppression of a selector codon in the negative marker, e.g., a gene that encodes a toxic protein, such as barnase. If the synthetase is able to charge the suppressor tRNA in the absence of unnatural amino acid, the cell will be killed by translating the toxic gene product. Survivors passing both selection screens encode synthetases that specifically charge the orthogonal tRNA with an unnatural amino acid.

The steps used in selection can include, e.g., a direct replica plate method. For example, after passing the positive selection step, cells can be grown in the presence of either ampicillin or chloramphenicol (depending on the negative selection marker being used) and in the absence of the unnatural amino acid. Those cells that do not survive are isolated from a replica plate supplemented with the unnatural amino acid. Compared to other potential selection markers, a positive selection based on antibiotic resistance offers the ability to tune selection stringency by varying the concentration of the antibiotic, and to compare the suppression efficiency by monitoring the highest antibiotic concentration at which cells can survive.

The foregoing selection steps, e.g., the positive selection step, the negative selection step or both the positive and negative selection steps in the above described-methods, optionally includes varying the selection stringency. For example, because barnase is an extremely toxic protein, the stringency of the negative selection can be controlled by introducing different numbers of selector codons into the barnase gene. In one aspect of the present invention, the stringency is varied because the desired activity can be low during early rounds. Thus, less stringent selection criteria can be applied in early rounds and more stringent criteria can be applied in later rounds of selection.

Other types of selections can also be used to produce O-RS, O-tRNA, and O-tRNA/O-RS pairs. In one embodiment, the positive selection step, the negative selection step, or both the positive and negative selection steps described above can include using a reporter detected by fluorescence-activated cell sorting (FACS). For example, a positive selection can be done first with a positive selection marker, e.g., a chloramphenicol acetyltransferase (CAT) gene, where the CAT gene comprises a selector codon, e.g., an amber stop codon, followed by a negative selection screen based on the inability to suppress a selector codon(s), e.g., two or more codons, at positions within a negative marker, e.g., the T7 RNA polymerase gene. In another embodiment, the positive selection marker and the negative selection marker can be found on the same vector, e.g. a plasmid. Expression of the negative marker drives expression of the reporter, e.g., green fluorescent protein (GFP). The stringency of the selection and screen can be varied, for example by varying the intensity of the light needed to excite the reporter (to observe a fluorescence signal). In another embodiment, a positive selection can be done with a reporter as a positive selection marker screened by FACs, followed by a negative selection screen based on the inability to suppress a selector codon at positions within a negative marker, e.g., a bamase gene.

Optionally, the reporter is displayed on a cell surface, e.g., in a phage display system. Cell-surface display, such as the OmpA-based cell-surface display system, relies on the expression of a particular epitope, e.g., a poliovirus C3 peptide fused to an outer membrane porin OmpA, on the surface of an E. coli cell [see, Francisco, J. A., Campbell, R., Iverson, B. L. & Georgoiu, G. Production and fluorescence-activated cell sorting of E. coli expressing a functional antibody fragment on the external surface. Proc. Natl. Acad. Sci. USA 90:10444-8 (1993)]. The epitope is displayed on the cell surface only when a selector codon in the protein message is suppressed during translation. The displayed peptide then contains the amino acid recognized by one of the mutant aminoacyl-tRNA synthetases in the library, and the cell containing the corresponding synthetase gene can be isolated with antibodies raised against peptides containing specific unnatural amino acids.

### Labeling Methods

The present methods of specifically incorporating an unnatural amino acid having a fluorophore into a protein can be carried out either using an in vitro translation system or in vivo using a cell. For example, when an O-tRNA/O-RS pair is introduced into a host cell, e.g. a CHO cell, in the presence of a growth medium containing the unnatural amino acid, the activity of the O-tRNA/O-RS pair results in the in vivo incorporation of the unnatural amino acid into a protein in response to a selector codon. Alternatively, the present compositions can be used in an in vitro translation system.

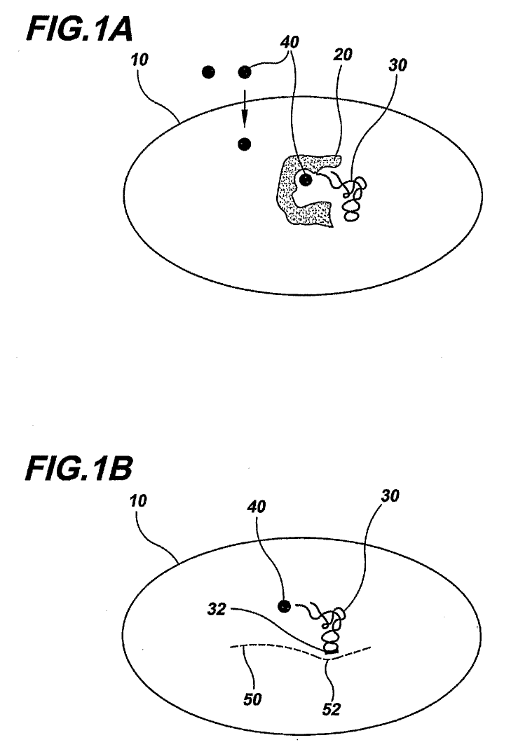

The site-specific incorporation of unnatural amino acids comprising fluorophores into proteins in vivo according to the present methods is schematically illustrated in FIGS. 1A-1D. A eukaryotic host cell 10 is provided, e.g., with an aminoacyl synthetase derived from L. lactis 20 and tRNA derived from L. lactis 30. The synthetase 20 aminoacylates the tRNA 30 with an unnatural amino acid comprising a fluorophore 40 which is introduced into the cell 10.

The cell 10 further comprises an mRNA molecule 50 having a selector codon 52. When a ribosome 60 encounters the selector codon 52 in the process of translating the mRNA molecule 50, the anticodon 32 of the tRNA 30 recognizes the selector codon 52 and the ribosome 60 catalyzes the formation of a peptide bond between the unnatural amino acid 40 and a natural amino acid 80 adjacent to it in the peptide chain of the protein 70 being formed. A full-length protein product is thus produced which includes the unnatural amino acid 40 incorporated therein.

In an alternative labeling strategy, an unnatural amino acid that comprises a reactive moiety can first be incorporated into a protein of interest during translation of the protein, and the reactive moiety can then be reacted with a fluorescent moiety in order to bind the fluorescent moiety to the precursor after it has been incorporated into the protein molecule. The reaction between the fluorescent moiety and the reactive moiety is preferably selective, i.e. such that the fluorescent moiety does not react non-specifically with other amino acid residues or moieties of a protein molecule. Such a reaction should also take place under conditions which do not damage or otherwise change the activity of the protein molecule, and more preferably can take place under physiological conditions, e.g. in vivo.

In one example of such a reaction, one or more keto-containing unnatural amino acids such as m-acetyl-L-phenylalanine or p-acetyl-L-phenylalanine can be selectively incorporated into a protein molecule, and these unnatural amino acids can then be labeled with hydrazide-containing fluorescent probes. In aqueous solution, the keto group of the unnatural amino acid reacts with hydrazide or alkoxyamine derivatives of the probes to form hydrazones or oximes, respectively (see, Zhang, Zhiwen, et al., A New Strategy for the Site-Specific Modification of Proteins in vitro, Biochemistry, 42: 6735-6746 (2003); Wang, L., et al., Addition of the keto functional group to the genetic code of Escherichia coli, PNAS, 100: 56-61 (Jan. 7, 2003); and Cornish, V. W., Hahn, K. M., and Schultz, P. G., Site-specific protein modification using a ketone handle. J. Am. Chem. Soc., 118: 8150-8151 (1996)). This reaction can be carried out not only in vitro but also in living cells.

In another embodiment, proteins into which have been incorporated unnatural amino acids having alkynyl and azido functional groups, such as para-propargyloxyphenylalanine, can be site-specifically labeled with fluorescent probes which include an azide moiety. Such labeling occurs through the irreversible formation of triazoles by a [3+2]cycloaddition in presence of copper(I) at room temperature in aqueous media (see, Deiters, A. and Schultz, P., In vitro incorporation of an alkyne into proteins in Escherichia coli, Bioorganic & Medicinal Chemistry Letters, 15:1521-1524 (2005)).

### Spectroscopic Methods

A variety of known instruments can be used to measure the fluorescence of fluorophores incorporated into a protein according to the present methods. Steady-state fluorescence can be measured, e.g., at room temperature using a Photon Technology International QM-1 fluorescence spectrophotometer equipped with excitation intensity correction and a magnetic stirrer. Suitable instrumentation for the present fluorescence polarization assays can include a fluorescence polarization plate reader for quantitative detection.

The excitation and emission spectra of fluorophores will vary depending on where the fluorophore(s) is incorporated into a particular protein. For measurements of NBD fluorescence, for example, emission spectra from 500 nm to 600 nm can be collected (λex. 470 nm, 0.1 to 1 second nm, bandpass 0.4 nm for excitation and emission). Fluorescence spectra of other fluorophores and other methods of detecting such emission spectra are known to or can be readily determined by those of skill in the art.

### Assay Targets

The present methods of incorporating a fluorescent moiety into a protein can be applied to a wide variety of proteins and thereby enable such proteins to be studied with greater accuracy. The present methods are particularly advantageous for the study of protein structure and function, since the unnatural amino acids having fluorescent moieties used in the present methods can be selected so as not to significantly alter the conformation of a protein to be studied, and since such amino acids can be precisely located at a location in the protein whose structure or function is to be evaluated (i.e. at a predetermined position in the amino acid sequence of the protein).

One class of proteins that can be advantageously studied using the present methods are enzymes, as an enzyme's activity is generally associated with its structure (conformation) and/or with its interaction with other molecules such as cofactors, activators, and inhibitors. Enzymes which can be analyzed according to the present methods include oxidoreductases, transferases, hydrolases, lyases, isomerases, and ligases. Examples of such enzymes include kinases, ATPases, phosphatases, phosphodiesterases, lipases, and proteases. Particularly preferred enzymes for analysis according to the present methods are those involved in producing clinical disease in humans or animals, for example as a result of the activation, inactivation, regulation, dysregulation, or other change or impairment in function of an enzyme of a human or animal. Such enzymes include those listed in Table 2 below.

TABLE 2
Enzymes

Oxidoreductases
Aldehyde dehydrogenase
Monoamine oxidases
(MAOs)
Cyclooxygenases (COXs)
Vitamin K epoxide
reductase
Aromatase
Lanosterol demethylase
Lipoxygenases
5-lipoxygenase
Thyroidal peroxidase
Iodothyronine-
5'deiodinase
Inosine monophosphate
dehydrogenase
HMG-CoA reductase
5α-Testosterone reductase
Dihydrofolate reductase
(bacterial)
Dihydroorotate reductase
Enoyl reductase
(mycobacterial)
Squalene epoxidase
(fungal)
Δ14 reductase (fungal)
Xanthine oxidase
4-Hydroxyphenylpyruvate
dioxygenase
Ribonucleoside
diphosphate reductase
Transferases
Protein kinase C
Bacterial peptidyl
transferase
Catecholamine-O-
methyltransferase
RNA polymerase
(bacterial)
Reverse transcriptases
(viral)
Allosteric inhibitors
DNA polymerases
GABA transaminase
Tyrosine kinases
Glycinamide
ribonucleotide formyl
transferase
Phosphoenolpyruvate
transferase
Human cytosolic
branched-chain
aminotransferase
Hydrolases (proteases)
Aspartyl proteases (viral)
Serine proteases
Bacterial lactamases
Human antithrombin
Human plasminogen
Human coagulation factor
Human factor Xa
Metalloproteases
Human ACE
Human HRD
Human carboxypeptidase
A (Zn)
Human enkephalinase
Esterases
Glycosidases
Lipases Gastrointestinal
lipases
Phosphatases
Inositol polyphosphate
phosphatase
GTPases
Phosphorylases
Threonine proteases
Aspartic proteases
Lyases
DOPA decarboxylase
Carbonic anhydrase
Histidine decarboxylase
Ornithine decarboxylase
Soluble guanylyl cyclase
Isomerases
Alanine racemase
DNA gyrases (bacterial)
Topoisomerases
Δ8,7 isomerase (fungal)
Ligases (also known as
synthases)
Dihydropteroate synthase
Thymidylate synthase
(fungal and human)
Phosphofructokinase
mTOR
Haem polymerase
(Plasmodium)
1,3-□-d-glucansynthase
(fungi)
Glucosylceramide
synthase

In one embodiment, the present methods can be used to evaluate enzymes that adopt different conformational states, such as conformational states associated with different degree of catalytic activity. For example, kinases, in particular protein kinases, adopt an “on” state that is maximally active, and an “off” state that has minimal activity. Fully active kinases are generally phosphorylated in their activation loop, which adopts a conformation that allows for optimal binding of ATP/Mg2+ and substrate protein, and for efficient transfer of the phosphate group of ATP to the protein substrate. The study of kinase conformation with the present methods can lead to improved treatments for diseases with which the improper activation of protein kinases have been implicated, including cancer, Alzheimer's disease, and type II diabetes. Kinases such as c-Src, c-Abl, mitogen activated protein (MAP) kinase, phosphotidylinositol-3-kinase (PI13K), AKT, and epidermal growth factor (EGF) receptor are commonly activated in cancer cells, and are believed to contribute to tumorigenesis.

The present methods can be used, for example, to identify kinase inhibitors, in particular inhibitors that bind outside the ATP-binding site. Fluorophores incorporated into kinases using the present methods are much less likely to disrupt protein function when compared to the use of fused or tethered large protein reporters, as used in prior art methodologies. Preferred fluorophores in this embodiment are environmentally-sensitive fluorophores whose fluorescent signal intensities differ when they are exposed to environments of different hydrophobicity and/or polarity. When such fluorophores are incorporated into a kinase molecule (or into another protein) in regions that transition from a “buried,” hydrophobic environment in one activation state (e.g., an inactive state) to an aqueous exposed environment in a second activation state (e.g., an active state), the conformation of such a molecule in the presence of a particular target molecule of interest can be studied by measuring changes in the fluorescent signal from the labeled protein. For example, if a molecule of NBD, which exhibits high fluorescence in a “buried” hydrophobic environment and low fluorescence in an exposed aqueous environment, is present in a hydrophobic environment at a particular location within the inactive conformation of the kinase and then becomes exposed to the aqueous environment when the kinase transitions to the active conformation, the fluorescence signal will decrease. If a target molecule binds to and stabilizes the inactive state of the kinase molecule, such stabilization can be detected by measuring an increase in the fluorescent signal when the kinase is in the presence of the target molecule as compared to when the target molecule is not present.

Other enzymes of interest which can be studied using the present methods include those of parasites, viruses and other infectious organisms and/or non-beneficial organisms, which can be evaluated in order to better understand how such enzymes function and preferably to determine how to inhibit or otherwise interfere with them. Examples of such enzymes include reverse transcriptases, integrases, proteases, and neuraminidase.

In another embodiment, the present fluorophore-containing amino acids can be incorporated into a protein believed to be involved in a disease state, and the present methods can be accomplished in vivo in a cell in order to allow the protein to be observed in vivo. In this case the targets can be, for example, proteins known or suspected to be involved in aggregation and/or which cause disease through misfolding. The present methods are particularly adapted to measuring aggregation and changes in protein conformation, both of which have been implicated in disease states in humans. Table 3 below lists some of the proteins and disease states known or believed to be involved in protein misfolding or aggregation.

TABLE 3
Proteins Involved in Aggregation or Misfolding

| Disease State | Protein Involved |
|---|---|
| Alzheimer's disease | Aβ peptide, 1-42, 1-43 |
| Spongiform encephalopathies | Full-length prion or fragments |
| Primary systemic amyloidosis | Intact light chain or fragments |
| Secondary systemic amyloidosis | 76-residue fragment of amyloid |
| | A protein |
| Familial amyloidotic polyneuropathy I | Transthyretin variants and |
| | fragments |
| Senile systemic amyloidosis | Wild-type transthyretin and |
| | fragments |
| Hereditary cerebral amyloid angiopathy | Fragment of cystatin-C |
| Hemodialysis-related amyloidosis | β₂₋microglobulin |
| Familial amyloidotic polyneuropathy II | Fragments of apolipoprotein A-1 |
| Finnish hereditary amyloidosis | 71-residue fragment of gelsolin |
| Type II diabetes | Fragment of islet-associated |
| | polypeptide |
| Medullary carcinoma of the thyroid | Fragments of calcitonin |
| Atrial amyloidosis | Atrial natriuretic factor |
| Lysozyme amyloidosis | Full-length lysozyme variants |
| Insulin-related amyloid | Full-length insulin |
| Fibrinogen a-chain amyloidosis | Fibrinogen a-chain variants |

### Assay Methods

The present compositions, systems, and methods can be used to evaluate a number of different features of a protein, including its structure, function, and interaction with other molecules. Such interactions include those with potential therapeutic agents as well as those with molecules which may cause disease, such as through aggregation or misfolding of the protein.

Protein Conformation Assays

In one embodiment, the present assays can be used to study conformational changes in a protein. In a preferred embodiment, environmentally-sensitive fluorophores are site-specifically incorporated into a protein, such as for example a kinase molecule, in order to study the protein, and in particular to study the interaction of a target molecule with the protein. Fluorophores that display minimum fluorescence intensity in aqueous media but undergo large increases in fluorescence intensity when transferred to more hydrophobic environments, such as nitrobenzoxadiazole (NBD), dansyl amino acid derivatives and acrylodan, are preferred in such assays, though it will be appreciated that fluorophores exhibiting a decrease in intensity when transferred to more hydrophobic environments can also be used. When such fluorophores are incorporated into a region of, e.g., a kinase which undergoes a conformational change when the kinase transitions from the inactive to the active state, such a transition can be monitored by measuring changes in the fluorescent signal of the fluorophore. For example, if the fluorophore is buried in a hydrophobic environment when the kinase is in the inactive state, and if the kinase undergoes a conformational change when transitioning to the active state so as to expose the fluorophore to an aqueous environment, a significant decrease in the fluorescent signal of the fluorophore will be observed. Such fluorophores are preferred because they have a very low fluorescent signal in aqueous media, so that free fluorophore will contribute a relatively small background signal as compared to the overall fluorescent signal.

It should be noted that the opposite approach can also be utilized. For example, if the fluorophore is exposed to an aqueous environment when the kinase is in the inactive state, and if the kinase undergoes a conformational change when transitioning to the active state so as to bury the fluorophore in a hydrophobic environment, a significant increase in fluorescence will be observed. In this embodiment, if the polarity-sensitive fluorophore is one whose signal strength decreases upon exposure to an aqueous environment, a weaker fluorescent signal is nonetheless generally emitted when the fluorophore is fluoresced, causing a background fluorescence in the assay. In order to reduce or eliminate this fluorescence and thereby increase the dynamic range of the fluorescent signal, a fluorescence quencher (e.g., iodide or cesium) can be added to the media containing the protein being assayed (either to an in vitro media or, in an in vivo assay, to the cell culture media). In the presence of such a quencher, the fluorescent signal emitted by the fluorophore when exposed to the aqueous media would decrease from a signal strength having a finite value when the fluorophore is buried in a first conformation to an undetectable or very low signal strength as the fluorophore becomes exposed and subsequently quenched when the protein assumes a second conformation. When iodide is used as the quencher, between 50-500 nM iodide is preferably added to a medium comprising the translation system being used, though other amounts (100 mM, 200 mM, 300 mM, 400 mM, etc.) can be used.

An alternative method for evaluating protein conformational changes involves the use of polarization fluorescence detection. Using the present methods, a fluorescent unnatural amino acid is incorporated at a single amino acid position in a protein which can exist in two different conformational states, as described above. The amino acid position is chosen such that the amino acid at the position undergoes a change in rotational mobility as the protein transitions from one conformational state to another conformational state. For example, a fluorophore incorporated at the desired amino acid position in a kinase experiences a restricted rotational mobility when the kinase is in the inactive conformation but experiences a larger degree of rotational mobility when the kinase is in the active conformation. The conformational change that occurs when the kinase transitions from the inactive to the active state would result in a decrease in the fluorescence polarization signal of the incorporated fluorophore.

Therapeutic or other agents can be evaluated using a labeled protein produced by the present methods. For example, a therapeutic agent can be added to a translation system (either an in vitro or in vivo system) which includes a labeled protein molecule as described herein, and the effect of adding the therapeutic agent can be monitored. If the fluorescent signal of the labeled protein in this example decreases upon transition from the inactive to the active state, a therapeutic agent that stabilizes the inactive or “off” state would prevent a decrease in the fluorescent signal. “Stabilization” of a protein by another molecule (target molecule), such as a therapeutic molecule, refers to the maintenance of the protein in a particular conformational state, such as an inactive conformational state, to a greater extent or for a longer period of time when in the presence of the target molecule than when in the absence of the target molecule. Agents which stabilize the protein in an inactive state can be identified as candidates for further development as therapeutic agents.

In one example of this embodiment, non-ATP competitive protein kinase inhibitors that preferentially stabilize the inactive conformation of the kinase are identified. In this example, the signals from small fluorophores that have been site-specifically incorporated into the kinase during protein translation are monitored, and compounds that allosterically inactivate protein kinases (i.e. stabilize the inactive or “off” state) are identified by monitoring the fluorescent signal of the incorporated fluorophore. Compounds that bind to and stabilize the inactive state can be identified by a lack of a decrease in fluorescent signal, for example, when the fluorophore used is NBD, the fluorophore is in a buried, hydrophobic environment in the inactive kinase, and activation of the kinase results in a conformational change which exposes the NBD molecule to a more aqueous environment. Conversely, compounds that bind to and stabilize the active state can be identified by a decrease in the fluorescent signal.

Using the present methods, a fluorescent unnatural amino acid is incorporated at a single amino acid position in a protein which can exist in two different conformational states. The amino acid position is chosen such that the amino acid at the position undergoes a change in rotational mobility as the protein transitions from one conformational state to another conformational state. For example, a fluorophore incorporated at the desired amino acid position in a kinase experiences a restricted rotational mobility when the kinase is in the inactive conformation but experiences a larger degree of rotational mobility when the kinase is in the active conformation. The conformational change that occurs when the kinase transitions from the inactive to the active state would result in a decrease in the fluorescence polarization signal of the incorporated fluorophore.

FRET Assays

In certain instances it may be desirable to increase the signal produced by a labeled protein. In this case, two or more unnatural amino acids comprising fluorophores can be incorporated at positions in the same region or in different regions of a protein in order to increase the fluorescent signal. A labeled protein produced by the present methods can include more than one environmentally sensitive fluorophore, for example at least two, three, or four fluorophores, at one or more regions in the kinase, in order to achieve an enhanced fluorescent signal.

Another method of achieving an enhanced optical signal in the present methods is through the use of Fluorescence Resonance Energy Transfer (FRET). In this embodiment, two fluorophores can be incorporated into the target protein and utilized to assess target protein aggregation, with one fluorophore serving as the donor and the other fluorophore as the acceptor. The two fluorophores in this case are positioned in a sufficiently close proximity to each other to allow a FRET interaction to occur, so that excitation of the donor fluorophore will result in an enhanced fluorescent emission from the FRET pair.

In addition to simply boosting an optical signal, FRET can be used to evaluate changes in the conformation of a protein. Two fluorophores (a FRET donor and acceptor) can be incorporated into a protein according to the present methods at locations in the protein such that a change in conformation of the protein will change the distance between the two fluorophores and result in a changed optical signal, i.e. the fluorescence signal of the acceptor fluorophore will be of greater intensity if the donor and acceptor fluorophores are moved closer together as a result of a conformational change and will be of lesser intensity if the donor and acceptor fluorophores are moved further apart.

### In Vivo Assays

One advantage conferred by the present methods is the ability to test whether small molecules act on a protein target, result in a desired phenotype, and lack undesired off-pathway effects in vivo. In one embodiment, an assay according to the present methods uses environmentally-sensitive fluorophores, e.g. nitrobenzoxadiazole (NBD) or ACRYLODAN, that display minimum fluorescence intensity in aqueous media but undergo large increases in fluorescence intensity when transferred to more hydrophobic environments (though it will be appreciated that fluorophores exhibiting a decrease in intensity when transferred to more hydrophobic environments can also be used). As the target protein-fluorophore undergoes aggregation, for example, the fluorophore becomes “buried” in a hydrophobic environment resulting in a significant increase in fluorescence signal. The increase in fluorescence signal serves as a measure of target protein aggregation. Given that the fluorophore has a very low fluorescent signal in aqueous media, free fluorophore and target protein-fluorophore monomer generally contribute a negligible background signal to the overall fluorescent signal.

Another method for controlling for background fluorescence in in vivo assays is the use of polarization fluorescence detection. When excited with polarized light, fluorophores attached to molecules having a high molecular weight (such as proteins) emit a high level of polarized fluorescence, since the rotation of such fluorophores is slower compared to that of fluorophores attached to smaller molecules (e.g., fluorophores bound to an unnatural amino acid). Detecting fluorescent emissions of labeled proteins using polarized light can therefore help to overcome problems with background fluorescence which may be caused by the presence of fluorescent moieties in unincorporated unnatural amino acids in cells.

Protein Interaction/Aggregation Assays

The present methods can also be used to monitor interactions between proteins, or between a protein and another type of molecule. In one embodiment, protein aggregation, such as that associated with Alzheimer's disease (AD), for example, can be evaluated. The extracellular plaques associated with AD result from the deposition of amyloid beta or Aβ peptide aggregates. Abeta peptides, including the most amyloid-genic form Aβ42, result from the proteolytic cleavage of amyloid precursor protein (APP) by β and β secretases.

An AD assay in this embodiment can comprise the use of a mammalian cell line that expresses NBD-labeled APP in which NBD is incorporated into the N-terminal region, residues 1-42, of APP (amyloid precursor protein). Aβ42-NBD can be formed by the cleavage of APP-NBD, and as monomeric Aβ42-NBD forms oligomers and eventually fibrils, the NBD fluorophore will become “buried” in a hydrophobic environment as the fibrils become associated with each other, resulting in a significant increase in fluorescence signal. The increase in fluorescence serves as a measure of Aβ42 aggregation. Given that NBD has a very low fluorescent signal in aqueous media, free NBD and Aβ42-NBD monomer will contribute a negligible background signal to the overall fluorescent signal.

Cells, such as HEK or CHO cells as well as neuronal cell lines (e.g. SYSH5), can be used in this embodiment. The cell lines can be used in compound screens for identification of molecules that prevent Aβ42 aggregation. Such an assay will allow for the screening compounds against of multiple targets in the cellular pathway from APP synthesis to Aβ42 formation and Aβ42 aggregation. Conditions involving other protein interactions, including other conditions involving protein aggregation, can likewise be evaluated in a similar manner, either in vivo or in vitro.

### EXAMPLES

### Example 1

### Identifying Orthogonal tRNA/RS Pairs and Hosts In Vitro

We prepared concentrated crude RS and total RNA from the following bacteria using published methods: Lactobacillus acidophilus, Lactobacillus casei, Lactococcus lactis, Gluconobacter oxydans and Rhodospirullum rubrum. As positive controls, we also isolated crude RS and total RNA from E. coli and Bacillus stearothermophilus (whose TyrRS/tRNA pairs have been shown to be orthogonal against mammalian TyrRS/tRNA pairs). We used commercially available bovine RS and total human tRNA.

Using the crude RS and RNA preparations, aminoacylation of Tyr tRNA was determined by measuring [³H]-Tyr incorporation in an in vitro assay. Reactions (60 μl) contained 50 mM Tris, 50 mM KCl, 2 mM DTT, 4 mM ATP, 2 mM Mg(OAc)₂, 0.3 nM [³H]-Tyr (54 ci/mmol), 10 μg total RNA preparation or 2 μg human tRNA and 25 μl concentrated crude bacterial RS preparation or 6-10 U bovine RS preparation. tRNA was omitted for control reactions. Following incubation at 37° C. for one hour, tRNA-[³H]-tyrosine was precipitated by transferring the reactions to tubes containing 3 ml ice cold 10% TCA and incubating them on ice for one hour. The precipitates were collected by vacuum filtration on GF/C filters presoaked with 10% TCA. Filters were washed three times each with 1 ml 10% TCA and two times with 1 ml ice cold EtOH and air-dried. Filter-retained radioactivity was determined by liquid scintillation counting.

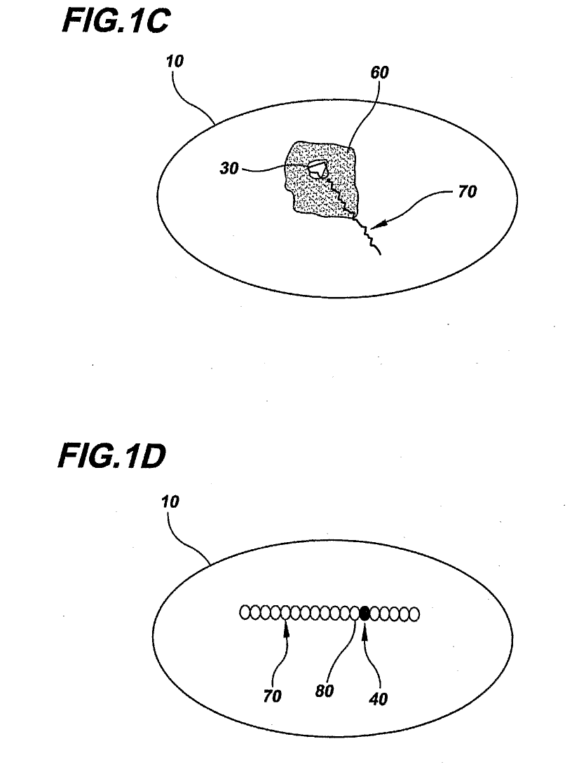

We first tested whether the bacterial Tyr tRNAs were orthogonal with respect to the bovine TyrRS (FIG. 2). The amount of radioactive TCA insoluble material collected on the filters represented tRNA-[³H]-Tyr. As expected, [³H]-Tyr incorporation above background was observed for reactions containing bovine RS and human RNA. As validation of our assay and consistent with published findings, we did not observe [³H]-Tyr incorporation in reactions containing E. coli and Bacillus stearothermophilus RNA. For the remaining bacterial RNA, with the exception of L. acidophilus, we did not observe any significant [³H]-Tyr incorporation above background. From these findings, we conclude that Tyr tRNA from L. casei, L. lactis, G. oxydans and R. rubrum are orthogonal with respect to mammalian TyrRS.

We next measured whether the bacterial TyrRS was orthogonal with respect to the bovine TyrRS. As validation of our assay and consistent with published findings, [³H]-Tyr incorporation was observed in reactions containing E. coli and B. stearothermophilus RS and their respective RNAs but not in reactions containing human RNA. For the remaining bacteria, with the exception of L. acidophilus and L. casei, we observed [³H]-Tyr incorporation above background only for reactions containing bacterial RS/bacterial RNA but not human RNA. From these findings, we conclude that TyrRS from L. lactis, G. oxydans and R. rubrum are orthogonal with respect to human tRNA.

### Example 2

### Amber Suppression in Mammalian Cells

We demonstrated the orthogonality and functionality of an O-RS/O-tRNA pair derived from L. lactis in a mammalian cell by rescuing an amber TAG mutation in the hERG potassium channel. In this experiment, human embryonic kidney (HEK) cells were transfected with cDNAs encoding the genes for hERG 652TAG, L. lactis “humanized” wildtype TyrRS (DNA: SEQ ID NO. 5, Protein: SEQ ID NO. 6) and modified, L. lactis Tyr amber suppressor tRNA_{CUA} (SEQ ID NO. 5). Protein expression was assessed by Western Analysis using an antibody specific for hERG. The results are summarized in Table 4 below.

TABLE 4
Suppression of hERG 652TAG Mutation

| | Lane 1 | Lane 2 | Lane 3 | Lane 4 | Lane 5 | Lane 6 | Lane 7 | Lane 8 |
|---|---|---|---|---|---|---|---|---|
| hERG WT | − | + | − | − | − | − | − | − |
| hERG | − | − | + | + | − | − | + | + |
| 652TAG | | | | | | | | |
| RS | − | − | − | − | + | − | + | + |
| tRNA_{CUA} | − | − | − | + | − | + | + | + |

As a positive control, HEK cells were transfected with wildtype hERG (Table 4, lane 2). HEK cells transfected with hERG 652TAG cDNA expressed hERG only when both the L. lactis RS and suppressor tRNA_{CUA} cDNAs were also transfected into the cells (Table 4, lanes 7 and 8). This finding clearly demonstrates that 1) the cells are expressing L. lactis tyrosyl RS and suppressor tRNA_{CUA}, 2) the L. lactis tyrosyl RS aminoacylates its tyrosyl suppressor tRNA_{CUA} and 3) the L. lactis tyrosyl suppressor tRNA_{CUA} aminoacylated with tyrosine can “rescue” the hERG 652TAG mutation.

Equally important is that no hERG expression was observed in cells transfected with hERG 652TAG and suppressor tRNA_{CUA} cDNAs (Table 4, lane 4). This indicates that the L. lactis suppressor tRNA_{CUA} is not aminoacylated by the endogenous human tyrosyl RS (i.e., this confirms orthogonality). The lack of hERG expression in cells transfected only with hERG 652TAG cDNA indicates that read-through by an endogenous tRNA is not occurring (Table 4, lane 3).

### Example 3

### A Mutant L. lactis TyrRS Library for Directed Evolution

To evolve a L. lactis O-RS specific for a particular unnatural amino acid (i.e., one comprising a fluorescent moiety), variants comprising all possible natural mutations are generated at key residues shown to be involved in amino acid binding. For a TyrRs, these key residues are: Tyr37, Asn123, Asp176, Phe177 and Leu180 [see, Brick, P, Bhat, T N, and Blow, D M, Structure of tyrosyl-tRNA synthetase refined at 2.3 A resolution, Interaction of the enzyme with the tyrosyl adenylate intermediate, J Mol Biol, 208:83-98 (1989)]. Further, directed evolution using a library of random mutations at these five positions can then be utilized in isolating mutants that charge such an unnatural amino acid [see, e.g., Chin, J W, Cropp, T A, Chu, S, Meggers, E, and Schultz, P G, Progress toward an expanded eukaryotic genetic code, Chem Biol, 10:511-519 (2003); Santoro, S W, Wang, L, Herberich, B, King, D S, and Schultz, P G, An efficient system for the evolution of aminoacyl-tRNA synthetase specificity, Nat Biotechnol, 20:1044-1048 (2002)].

To generate a library encoding all possible mutations at positions 37, 123, 176, 177 and 180, overlapping PCR primers and oligonucleotides that contain degenerate codons corresponding to these positions are used. In this way a final PCR product coding for full-length TyrRS that contains 3.2×10⁶ individual mutants is generated. The final PCR product(s) are then subcloned into the BamHI/EcoRI sites of ptRNA_{CUA}/ADH1 (described below) to yield a mutant library (ptRNA_{CUA}/ADH1-mutRS).

### Example 4

### Yeast Selection System for Isolating Mutant L. lactis TyrRS Molecules

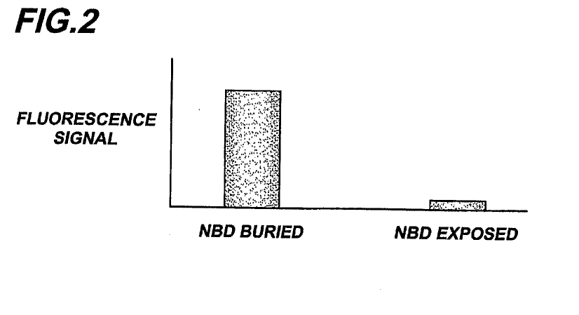

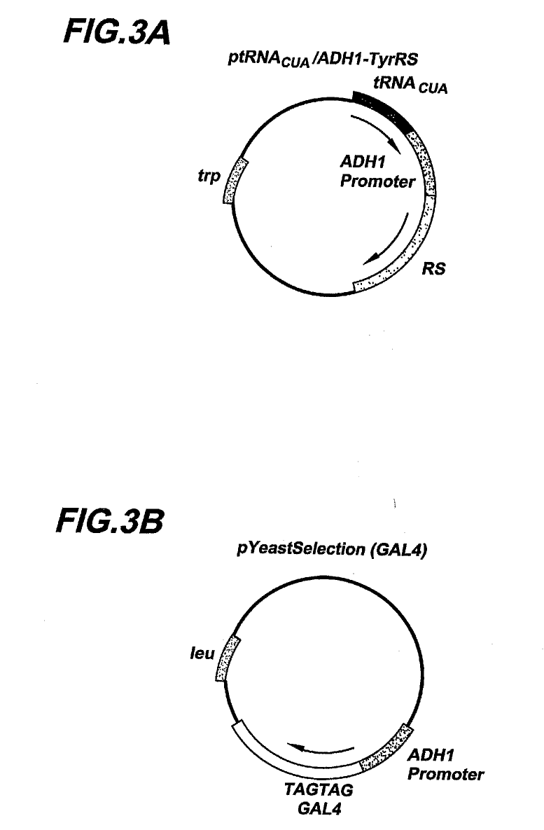

A yeast screening system is used for isolating L. lactis TyrRS mutants that specifically recognize unnatural amino acids, i.e. fluorescent unnatural amino acids. This approach was used to select E. coli TyrRS mutants [see, Chin, et al., Progress toward an expanded eukaryotic genetic code, Chem Biol 10:511-9 (2003)]. Two plasmids are transformed into the yeast cells in this system in order to isolate mutant L. lactis TyrRS. One is a plasmid selected from a plasmid library containing suppressor tRNA_{CUA} and TyrRS mutants (ptRNA/ADH1-TyrRS, shown in FIG. 3A) and the other is a plasmid containing the GAL4 gene that has two TAG mutations (pYeastSelection, shown in FIG. 3B).

To generate ptRNA/ADH1-TyrRS, the L. lactis suppressor tRNA_{CUA} construct (SEQ ID NO:2) designed for expression in mammalian cells, comprising 5′ and 3′ UTR regions of the human Tyr tRNA gene, was modified, as it has been reported that human tRNA genes do not generally express well in yeast unless the 5′ and 3′ UTRs are replaced. Through PCR, the L. lactis suppressor tRNA_{CUA} construct was modified to contain the 5′ and 3′ UTRs from the yeast Tyr tRNA gene (SEQ ID NO:3).

The L. lactis TyrRS (SEQ ID NO:10) and tRNA_{CUA} genes were subcloned into the yeast expression vector pESC-TRP (Stratagene). To drive the expression of TyrRS, we inserted the yeast ADH1 promoter immediately upstream of the TyrRS gene which was subcloned using restriction sites engineered into the gene. We generated restriction enzyme sites for the ADH1 promoter through PCR, and restriction enzyme sites for the tRNA_{CUA} gene were introduced with the PCR to modify the 5′ and 3′ UTRs. The DNA fragments and pESC-TRP were digested with the appropriate restriction enzymes, and the fragments were ligated to generate ptRNA_{CUA}/ADH1-TyrRS (FIG. 3A). Yeast transformed with ptRNA_{CUA}/ADH1-TyrRS can be selected by growing on media lacking tryptophan

To select for L. lactis synthetases specific for unnatural amino acids, we also generated a plasmid containing GAL4 that has two TAG mutations at positions 44 and 110. These two amino acid positions are permissive with respect to incorporation of a large variety of amino acids [see, Chin, et al., Progress toward an expanded eukaryotic genetic code, Chem Biol 10:511-9 (2003)], though they are not the only two positions that can be utilized. To generate pYeastSelect we isolated the yeast GAL4 gene from pCL1 by digestion with HindIII. The GAL4 HindIII fragment was then subcloned into the HindIII site of pGADGH. The TAG mutations at positions 44 and 110 were generated by site-directed mutagenesis (QuickChange). Yeast transformed with pYeastSelection can be selected by growing on media lacking leucine.

The yeast strain MaV203 is then transformed with each of the plasmids described above and grown in the presence of an unnatural amino acid in order to select for RS molecules which charge tRNAs with the unnatural amino acid. MaV203 has been engineered such that the transcription factor encoded by the GAL4 gene product has been knocked out and the genes encoding proteins required for the biosynthesis of uracil (URA3), histidine (HIS3), and β-galactosidase (LacZ) are under control of the GAL4 promoter. Yeast expressing a functional GAL4 transcription factor will grow on media lacking uracil or histidine. Functional mutant RS molecules aminoacylate the suppressor tRNA_{CUA}, resulting in rescue of the GAL4TAG mutant and expression of functional GAL4, which then drives the synthesis of the URA3 and HIS3 gene products.

Positive selection is then performed by growing yeast on media which lacks uracil, or on histidine-lacking media that contains 20 mM 3-amino-1,2,4-triazole, and which contains the unnatural amino acid. This selects for RS molecules that use natural amino acids, unnatural amino acids, or both. Only those yeast that express a functional mutant RS (using either a natural or the unnatural amino acid) will survive.

Negative selection is then performed by growing the surviving yeast on media containing 5-fluoroorotic acid (5-FOA) in the absence of the unnatural amino acid. The URA3 gene product converts 5-FOA to the toxic 5-fluorouracil, which causes yeast death and thereby selects for RS molecules that use only the unnatural amino acid. The surviving yeast are those that express a functional mutant RS synthetase that uses the unnatural amino acid. After two to three rounds of positive/negative selection, the plasmids containing the mutant RS are isolated from the surviving yeast.

### Example 5

### Synthesis of NBD-DAP

To a stirred solution of sodium bicarbonate (823 mg) and (S)-3-amino-2-(tert-butoxycarbonylamino)propanoic acid (1 g) in a 1:1 water and acetonitrile mixture was added 4-chloro-7-nitrobenzofurazan (NBD-Cl, 400 mg). The reaction was allowed to stir one hour before another 576 mg of NBD-Cl was added to the reaction. The reaction was allowed to stir for 48 hours. Solvent was removed using rotorary evaporation, and the remaining crude was dissolved in ethyl acetate and the product was extracted using a saturated bicarbonate solution. The saturated bicarbonate fractions were combined and neutralized with concentrated acetic acid until a pH of 5-6 was achieved. The product, Boc-NH-DAP-NBD-COOH, was extracted out of the aqueous solution using ethyl acetate. The combined ethyl acetate layers were dried over sodium sulfate, filtered, and the solvent removed by rotary evaporation. ¹H NMR (300 MHz, acetone-d6) δ 8.58 (d, J=8.7 Hz, 1H), 8.25 (bs, 1H), 6.37 (d, J=8.7 Hz, 1H), 6.66 (bs, 1H), 4.67 (m, 1H), 4.10 (m, 2H), 1.35 (s, 9H).

BOC-NH-DAP-NBD-COOH (572 mg) was dissolved in 6 ml of methylene chloride and 6 ml of TFA and stirred at room temperature for 2.5 hours. TFA and methylene chloride were removed by rotary evaporation. The product, NH₂-DAP-NBD-COOH, was dissolved in 25 ml of water and lyophilized overnight. ¹H NMR (300 MHz, dimethyl-d6 sulfoxide) δ 9.25 (bs, 1H), 8.60 (d, J=8.7 Hz, 1H), 8.44 (bs, 2H), 6.54 (d, J=8.7 Hz, 1H), 4.30 (m, 1H), 3.98 (m, 2H).

### Example 6

### Generation of O-RS/tRNA Pair Charging NBD-DAP

A mutant tyrosyl O-RS cDNA library is generated which is degenerate at the following five codons involved in tyrosine binding: Tyr34, Asn123, Asp176, Phe177 and Leu180. This library is then subjected to directed evolution in a S. cerevisiae yeast system, as described above. The directed evolution experiments are performed by transforming the yeast with (1) the suppressor tRNA_{CUA} cDNA (SEQ ID NO:3), (2) the mutant O-RS cDNA library, and (3) a cDNA containing a marker gene (GAL4 transcription factor) that contains nonsense TAG codons. Yeast cells harboring a functional O-RS and tRNA_{CUA} will “rescue” expression of the marker gene protein, i.e. by allowing them to grow on media lacking uracil or histidine.

In this experiment, both positive and negative selection steps are used to detect the presence of the marker gene protein. In the positive selection step, yeast are grown in the presence of NBD-DAP. Yeast harboring functional RS that use both NBD-DAP and any natural amino acid (primarily tyrosine) rescues expression of the marker gene protein and survives on selective media that requires the marker gene protein for survival. In the negative selection step, yeast are grown in the absence of NBD-DAP. Yeast harboring functional RS that use any natural amino acid (primarily tyrosine) will rescue expression of the marker gene protein and then die on selective media in which expression of the marker gene protein results in non-viability. Yeast surviving the negative selection step harbor mutant RS molecules that only recognizes NBD-DAP.

### Example 7

### NBD-Amino Acid Toxicity Testing

To show that NBD-DAP is taken up by CHO cells and that NBD-DAP is not toxic to the cells, CHO cells were incubated with 0.4 mM NBD-DAP for two days. After two days the cells were confluent. The cells were seen to have elongated bodies and few dead cells could be seen in the culture. The cells were washed several times with phosphate buffer to remove all NBD-DAP in the media and were then pelleted and lysed. The lysate was then analyzed using LC-MS. NBD-DAP was seen only in the lysate of CHO cells incubated with the unnatural amino acid. According to the mass spectral data, the cellular concentration of NBD-DAP in CHO cells incubated with unnatural amino acid is comparable to the cellular concentration of tyrosine and phenylalanine found naturally in these cells.

### Example 8

### Fluorescence of NBD In vivo

Another experiment was performed to see if fluorescence could be observed in CHO cells incubated with NBD-DAP when the cells were exposed to a hydrophobic environment. CHO cells were incubated with NBD-DAP for two days and exposed to a hydrophobic solution. The fluorescent signal from NBD-DAP at 521 nm was easily seen using a fluorescent microscope. No emission was seen between 495 to 602 nm in CHO cells not incubated with NBD-DAP.

### Example 9

### Assaying the Activation State of hAbl using NBD-hAbl

The activation state of hAbl (human v-abl Abelson murine leukemia viral oncogene homolog), a non-receptor tyrosine kinase, is assayed using the present methods. The activation loop, located within the active site of the kinase domain, contains an N-terminal Asp-Phe-Gly motif which serves to coordinate the Mg²⁺ associated with bound ATP. In the inactive state, the Asp-Phe-Gly motif is orientated in a suboptimum position for efficient transfer of a phosphate group from ATP and the residues C-terminal to the Asp-Phe-Gly motif block substrate binding by mimicking the conformation of the peptide substrate. Upon auto-phosphorylation of Tyr412 in the activation loop, the Asp-Phe-Gly motif undergoes a 180° C. flip, resulting in optimum Mg²⁺-ATP and peptide substrate binding.

cDNA clones. cDNA clones for hAbl (EX-T1921-B01; 1b variant) are obtained from Open Biosystems. A hexa-histidine (His₆) sequence and TAA stop codon are introduced just downstream of the kinase domain of hAbl by site-directed mutagenesis (QuickChange, Stratagene). The hAbl-His₆ gene is subcloned into the mammalian expression vector pcDNA3.1 (Invitrogen) to yield the plasmid hAbl-His₆:pcDNA3.1.

Expression and purification of hAbl kinase domain in mammalian cells. hAbl-His₆ is expressed in CHO cells following published procedures (Brasher, 2000). Cells are transfected with hAbl-His₆:pcDNA3.1 using LIPOFECTAMINE reagent available from Invitrogen. To isolate hAbl-His₆ in the inactive, non-phosphorylated state, transfected cells are grown in the presence of 50 μM GLEEVEC imatinib mesylate (Toronto Research Chemicals, N. York, Canada). 48-60 h post-transfection, cells are washed with phosphate-buffered saline containing 5 mM EDTA followed by solubilization (4° C., 15 min) in 0.5% Triton X-100, 20 mM Tris-HCl, pH 8.0, 150 mM NaCl, 5% glycerol, 5 mM 2-mercaptoethanol, 0.1 mM EGTA and protease inhibitor cocktail (Sigma). Following centrifugation (13,000×g, 20 min, 4° C.), cobalt nitrilotriacetic acid-agarose (Clontech) is added to the supernatant, mixed at 4° C. for 30 minutes and transferred to a 5 ml disposable column. The column is washed with 5 volumes of solubilization buffer, 2.5 volumes of wash buffer (20 mM Tris, 10 mM imidazole, pH 8.0, 150 mM NaCl, 0.05% Brij35, 0.1 mM EGTA and protease inhibitors) and 2.5 volumes of the same wash buffer containing 20 mM imidazole. OD₂₈₀ of wash fractions are measured to insure that all unbound proteins have been eluted. hAbl-His₆ is eluted with 2.5 volumes of wash buffer containing 100 mM imidazole. EDTA and dithiothreitol are added to final concentrations of 2 and 1 mM, respectively, and the fraction concentrated in a Centricon YM10 concentrator (5,000×g, 4° C., 1 hr). The concentrated fraction (˜50 μl) is diluted 40-fold with 100 mM Tris-HCl, pH 7.5, 100 mM NaCl, 0.2 mM EDTA, 0.02% Brij35, and 2 mM dithiothreitol and re-concentrated (repeated two times). To remove any active, phosphorylated hAbl-His₆, agarose-linked anti-phosphotyrosine antibody (#525300, Calbiochem, San Diego, Calif.) is added to the final concentrated hAbl-His₆ fraction and incubated with gentle shaking (4° C., 2 hrs). The anti-phosphotyrosine-agarose beads are removed by centrifugation (13,000×g, 4° C., 30 min). An equal volume of 100% glycerol is added and the purified hAbl-His₆ solution stored at −20° C.

hAbl-His₆ purity is assessed by SDS-PAGE and concentration determined by Bradford assay (Pierce). To verify that the purified hAbl-His₆ is in the inactive, non-phosphorylated state, Western analysis is performed using antibodies that recognize the inactive, non-phosphorylated state and active, phosphorylated state (ab4717, ab15130, available from Abcam, Inc., Cambridge, Mass.).

hAbl kinase activity assay. Activity of hAbl-His₆ is monitored by combining an auto-phosphorylation assay (to fully activate the kinase) and a spectrophotometric kinase assay as described (Barker, 1995). The kinase assay measures the consumption of ATP coupled to the oxidation of NADH (as observed by a decrease in absorption at 340 nm) via pyruvate kinase/lactate dehydrogenase. Reactions (50 μl) consist of 100 mM Tris (pH 8.0), 10 mM MgCl₂, 2 mM dithiothreitol, 1 mM EGTA, 0.01% Brij35, 2.2 mM ATP, 1 mMphosphoenolpyruvate, 0.6 mg/mL NADH, 75 U/mL pyruvate kinase, 105 U/mL lactate dehydrogenase (P0294, Sigma) and 0.5 mM Abl kinase substrate peptide (sequence: EAIYAAPFAKKK; Sigma). The initial OD₃₄₀ value is measured and the reaction initiated by the addition of 30 nM purified hAbl-His₆. Blank reactions containing no peptide substrate are run to assess the consumption of ATP by auto-phosphorylation of hAbl-His₆. The decrease in OD340 is measured after 30 minutes incubation at 30° C. The levels of auto-phosphorylation are determined by Western analysis using antibodies that recognize the non-phosphorylated and phosphorylated forms of hAbl.

Preparing NBD-hAbl. The unnatural amino acid NBD-DAP is site-specifically incorporated into hAbl using the present methods at to produce NBD-hAbl, as set forth below.

The following residues lie at the interface between the SH2, SH3 and kinase domains in the inactive state: Ser152, Asn154, Ala156, Leu321 Leu360, Tyr361, and Cys483. NBD-DAP incorporated at each of these residues is buried in a hydrophobic environment in the inactive state and experiences an increase in exposure to aqueous media upon transitioning from the inactive to the active state.

Another region that has been shown to undergo a change in conformation upon activation is the highly conserved activation loop region. Upon phosphorylation of Tyr412, the activation loop undergoes a conformational change that allows for ATP/Mg²⁺ and peptide substrate binding. NBD-DAP can also be incorporated in hAbl at individual positions located within the activation loop (Leu403, Thr411, Trp424, Phe420).

NBD-labeled hAbl is generated using the O-RS/O-tRNA pair described above. CHO cells are transfected with plasmids encoding this O-RS/O-tRNA pair and hAbl TAG mutant cDNA (i.e., a hAbl-His₆:pcDNA3.1 plasmid having an hAbl sequence into which a TAG stop codon has been introduced in place of one of the codons) and are grown in the presence of NBD-DAP (1 mM). NBD-DAP enters the cells, and the O-RS aminoacylates the O-tRNA with NBD-DAP. The aminoacylated tRNA delivers NBD-DAP into the desired position of hAbl. For each of the positions listed above, NBD-labeled hAbl is purified and Western analysis is used to assess the efficiency of incorporation of NBD-DAP into hAbl. Functional assays are performed to ensure that NBD incorporation does not alter kinase activity.

Assaying Inactive and Active States of hABL using NBD-hABL. NBD in the NBD-hAbl molecules described above is less accessible to the aqueous environment in the inactive state of such kinase molecules, and such molecules therefore display a larger fluorescence signal in the inactive state than in the active state, as illustrated in FIG. 1. As NBD-hAbl conformation transitions from the inactive state to the active state, NBD becomes more exposed to the aqueous environment, resulting in a decrease in fluorescence signal.

Measurement of fluorescence properties of NBD-hAbl. Fluorescence intensity and excitation/emission spectra of each of the NBD-hAbl-His₆ proteins in the inactive, non-phosphorylated state and, following auto-phosphorylation, in the active state are measured using a Molecular Devices Spectra Max Gemini XS fluorescence plate reader, including emission spectra over a wavelength range of 500-800 nm (λₑₓ=470 nm) and excitation spectra over a wavelength range of 300-520 nm (λₑₘ=570 nm). NBD-hAbl-His₆ proteins for use in the present assays display a larger fluorescence signal (increased quantum yield) when in the inactive state and have blue-shifted excitation and emission spectra relative to that observed following transition to the active conformation. Recovery of a fluorescent signal following treatment of active NBD-hAbl with YOP phosphatase is also measured. Those NBD-hAbl proteins displaying a measurable difference in fluorescence signal for the inactive and active conformational states are useful in the present methods.

Iodide quenching experiments are also performed to ascertain the degree of NBD exposure in the inactive and active conformational states. Fluorescence intensity of NBD-hAbl-His₆ proteins is measured as a function of iodide concentration (0-500 mM). Solutions containing non-phosphorylated and auto-phosphorylated NBD-hAbl proteins (100 mM Tris, pH 8.0, 10 mM MgCl₂, 2 mM dithiothreitol, 1 mM EGTA) are titrated with a potassium iodide solution (containing 0.1 mM thiosulfate to prevent iodide oxidation) and the fluorescence intensity measured (fluorescence intensity is corrected for dilution). Stern-Volmer plots (F₀/F vs [I⁻]) are generated and Stern-Volmer quenching constants (K_{SV}) calculated. Quenching of NBD fluorescence increases upon auto-phosphorylation of NBD-hAbl.

Validating fluorescence-based screen. To validate that the present assay detects compounds that bind to and stabilize the inactive hAbl conformation, changes in NBD-hAbl-His₆ fluorescence following auto-phosphorylation/activation in the presence of increasing concentrations of GLEEVEC are measured. Stabilization of the inactive conformation of NBD-hAbl-His₆ by GLEEVEC prevents activation and, in turn, results in a decrease in fluorescence intensity.

Auto-phosphorylation assays are performed in the presence of increasing concentrations of GLEEVEC ranging from 0-200 nM (reported IC₅₀ value=25 nM ref). NBD-hAbl-His₆ proteins at a concentration of at least 1 μM are incubated with GLEEVEC for 15 minutes. Reactions are initiated by the addition of NBD-hAbl/GLEEVEC to the auto-phosphorylation buffer (50 mM Tris-HCl, pH 7.5, 10 mM MgCl₂, 2 mM dithiothreitol, 1 mM EGTA, and 0.01% Brij35, 500 μM ATP). Fluorescence intensity is measured in real-time to determine the time course for maximal changes in fluorescence intensity. Fluorescence intensity is plotted as a function of GLEEVEC concentration and the IC₅₀ calculated by fitting the dose-response relationship to the Hill equation (F=Fmax/(1+(IC50/[GLEEVEC]^{nH})). For comparison, the IC₅₀ for GLEEVEC is determined. Following GLEEVEC inhibition, Western analysis is performed using antibodies that recognize the non-phosphorylated and phosphorylated states to confirm that GLEEVEC inhibits hAbl activity by preventing hAbl auto-phosphorylation.

### Example 10

### In vivo Labeling with Fluorescein Hydrazide

Instead of labeling hAbl with NBD-DAP as in Example 9, hAbl can instead be labeled with m-acetylphenylalanine. In this case, CHO cells comprising an O-RS that can charge an O-tRNA with m-acetylphenylalanine are first prepared according to the present methods. A suspension of such CHO cells (100 mL) are then pelleted and washed with 1×PBS (pH 7.0) containing 10% glycerol, to remove excess m-acetylphenylalanine. The cells are resuspended in mL of 1×PBS containing 10% glycerol. Fluorescein hydrazide (4.5 mg, 1 mM) is dissolved in 500 μL of DMF and added dropwise to the cell culture suspension. Additional 1×PBS buffer (pH7.0) containing 10% glycerol is then added to bring the final volume to 10 mL. The reaction mixture is gently tumbled at 4° C. for 18 h. Cells are harvested and washed thoroughly with 1×PBS buffer (pH 7.0).

### Example 11

### Enhancing Fluorophore Signal Strength

In Example 9, prior to measuring the fluorescence properties of NBD-hAbl following auto-phosphorylation/activation in the presence of increasing concentrations of GLEEVEC, 400 mM iodide is first added to the medium as a quencher. The differential signal strength measured for the different conformations of NBD-hAbl is increased.

### Example 12

### Development of a DANSYL-O-RS/O-tRNA Pair

The yeast strain MaV203 was transfected with plasmids coding for the DANSYL-O-RS (SEQ ID NO:12, derived from E. coli), suppressor O-tRNA (SEQ ID NO:5 derived from E. coli) and a marker gene, GAL4, containing nonsense TAG codons. Yeast harboring a functional DANSYL-RS_{OR} and suppressor tRNA OR will “rescue” expression of GAL4. The MAV203 yeast strain contains the URA3 gene (required for uracil biosynthesis) under control of the GAL4 promoter. When grown in the absence of uracil, MAV203 yeast will not grow unless “rescue” of GAL4 expression occurs. Growth was only observed when the transformed yeast were grown in the presence of dansyl-alanine. This data demonstrates that this DANSYL-O-RS/O-tRNA pair is capable of incorporating a danysl fluorophore into GAL4. Further, given that we did not observe growth in the absence of dansyl-alanine indicates that the DANSYL-O-RS/O-tRNA pair is selective for dansyl-alanine.

We next demonstrated the ability to incorporate dansyl-alanine into proteins expressed in mammalian cells. We first redesigned the suppressor O-tRNA gene to make it suitable for expression in mammalian cells by altering the 5′ UTR, mutating the promoter regions and generating a tandem repeat, thereby forming SEQ ID NO:6, and subcloned the O-RS gene (SEQ ID NO:12) into a mammalian expression vector. We transformed CHO cells with the plasmids encoding the modified O-RS/O-tRNA genes and a marker gene, Chloramphenicol Acetyltransferase (“CAT”), containing a nonsense TAG codon (“CAT-TAG”). Expression of CAT-TAG was assessed using a fluorescent-based assay (FAST CAT, Invitrogen, Inc.) that measures the conversion of chloramphenicol-BODIPY (FL-CM) to 3-acetyl-chloramphenicol-BODIPY (FL-AcCM) by thin layer chromatography. Transfected CHO cells grown in the presence of dansyl-alanine expressed significant CAT activity, indicating the incorporation of dansyl-alanine into CAT-TAG in mammalian cells. Cells transfected only with CAT-TAG cDNA did not display any significant CAT activity. As a positive control, cells were transfected with wildtype CAT cDNA.

### Example 13

### Fluorescence Polarization for Measuring Dimerization

A high-throughput fluorescence-based assay that can be used to map protein-protein interactions in cellular pathways and to screen for small-molecule protein-protein interaction modulators is developed as follows, utilizing FK506-mediated dimerization of FKBP12 with calcineurin (CN) as a model system.

Establishing the FK506-FKBP12-calcineurin expression system. FKBPs (FK506 binding proteins) are rotomases and most likely function as molecular chaperones where they facilitate the intracellular transport of proteins and stabilize multiprotein complexes. FK-506, a bacterial toxin that has immunosuppressive activity, inhibits the enzymatic activity of the calcium/calmodulin-dependent phosphatase calcineurin (CN) by inducing the dimerization of FKBP12 with CN. cDNA clones for hFKBP12, hCNa and hCNb subunit are obtained from Open Biosystems (Huntsville, Ala.). hFKBP12 is subcloned into the mammalian expression vector pcDNA3.1. A nucleotide sequence coding for hexa-histidine (6×His) is inserted immediately after the C-terminus using standard site-directed mutagenesis techniques (QuickChange II, Stratagene, La Jolla, Calif.). hCNa and hCNb cDNAs are both subcloned into the bicistronic mammalian expression plasmid pIRES (Invitrogen, Carlsbad, Calif.). Subcloning both cDNAs into the same plasmid insures that all transfected cells will contain both hCNa and hCNb genes and, therefore, express both proteins.

Expression of hFKBP12, hCNa and hCNb in mammalian cells. CHO cell cultures are grown at 37° C. and 5% CO₂ in Ham's F12 media, enriched with glutamine, 10% fetal bovine serum, penicillin and streptomycin. Cells are passaged 24-36 hours prior to transfection at which time the cultures are 60-80% confluent. Cells are initially transfected with 2 μg per 1×10⁶ cells of both hFKBP12:pcDNA3.1 and hCNa/hCNb:pIRES using Lipofectamine 2000 (Qiagen, Valencia, Calif.). 48 hours post-transfection, protein are isolated using CelLytic M Cell Lysis Reagent (Sigma, St. Louis, Mo.) and expression levels of hFKBP12, hCNa and hCNb determined by Western analysis using the WesternBreeze kit (Invitrogen, Carlsbad, Calif.). Primary antibodies against hFKBP12, hCNa and hCNb are purchased from AbCam (Cambridge, Mass.). Mock transfections are performed to determine the levels of endogenous FKBP12 levels of CHO cells.

Determining sites of BODIPY-AA incorporation into hFKBP12. BODIPY is incorporated at 20 positions throughout hFKBP12. Since an unnatural amino acid comprising BODIPY is hydrophobic, incorporation sites that have hydrophobic amino acids (e.g. Val, Leu, Ile) are chosen. Using standard site-directed mutagenesis techniques, hFKBP12 pcDNA 3.1 constructs containing TAG codons at positions Val3, Val5, Phe16, Val25, Leu31, Phe37, Phe47, Val56, Trp60, Val64, Val68, Leu74, Ile76, Ile91, Ile92, Val98, Val101, Leu103, Leu104 and Leu106 are generated.

Site-specific incorporation of BOPDIPY-AA into hFKBP12. BODIPY-hFKBP12 protein is generated using an orthogonal mutant BODIPY-AA aminoacyl synthetase (RS)/suppressor tRNA expression system. CHO cells are grown in black-lined six well plates to minimize fluorescence bleed-through between adjacent wells and are transfected with plasmids encoding the genes for our orthogonal BODIPY RS/suppressor tRNA and one of the hFKBP12 TAG mutant described above and grown in the presence of 1 mM the BODIPY-AA, and Western Analysis using anti-hFKBP12 and/or anti-His₆ antibodies is used to assess the efficiency of incorporation of BODIPY-AA into hFKBP12 in each of these mutants. BODIPY-hFKBP12 proteins that do not express or express poorly are discarded. To minimize the amount of unincorporated BODIPY-AA present in the cells, the culture media is replaced with media lacking BODIPY-AA 24 hours prior to making fluorescence measurements. The cells are also washed three times with PBS immediately before taking measurements.

Measurement of BODIPY-hFKBP12 fluorescence properties and Fpol (fluorescence polarization value). For cellular imaging measurements, a TECAN Infinite Safire 2 Microplate Reader is used. Successful incorporation of BODIPY-AA into hFKBP12 is indicated by a reliably detectable FPol signal sufficiently above background signals. The fluorescence excitation and emission spectra (to determine the excitation/emission maxima) and the FPol value of each BODIPY-hFKBP12 is measured, and the FPol value is compared to that observed for free BODIPY (˜30 mP).

Characterization of fluorescence properties of BODIPY-hFKBP12. Free BODIPY has excitation and emission maxima of 493 nm and 503 nm, respectively. To determine whether these parameters change when BODIPY is incorporated into hFKBP12, the fluorescence excitation and emission maxima of BODIPY-AA and BODIPY-hFKBP12 are determined. Using the excitation/emission maxima determined from the spectra, the FPol value of BODIPY-hFKBP12 proteins expressed in CHO cells is measured using a TECAN InfiniteM200 plate reader. To determine the FPol value, the vertically (V) and horizontally-polarized (H) emitted light following excitation is measured with vertically-polarized light (designated I_{VV} and I_{VH}, respectively). Given that the efficiency of detecting vertically and horizontally polarized light is not equivalent the correction factor, G, is applied to I_{VH}. G is determined by measuring horizontally and vertically-polarized emitted light following excitation with horizontally-emitted light (I_{HV} and I_{HH}, respectively) and is given by the ratio I_{HV}/I_{HH}. The equation FPol=(I_{VV}−G*I_{VH})/(I_{VV}+G*I_{VH}) is used for calculating the FPol value. For each measurement the total fluorescence intensity (F=I_{VV}+2*G*I_{VH}) is also calculated to monitor the stability of BODIPY-hFKBP12.

Measurement of FK506-mediated BODIPY-hFKBP12 dimerization with hCN. The BODIPY-hFKBP12 proteins showing suitable expression levels are assessed for their ability for measuring FK506-mediated changes in FPol when co-expressed with hCN.

Determination of FK506 EC₅₀ values. Cells are exposed to FK506 one hour prior to FPol measurements. FK506 EC₅₀ values are determined for the BODIPY-hFKBP12 proteins displaying measurable FK506-dependent increases in FPol values when co-expressed with hCN. FPol measurements are made in cells co-expressing BODIPY-hFKBP12 and hCN following exposure to FK506 concentrations ranging from 0.1-300 nM. Base-line values consist of measurements made in the absence of FK506 and in cells expressing only BODIPY-hFKBP12. The EC₅₀ for FK506-mediated hFKBP12-hCN dimerization are calculated by fitting the data to the equation: FPol=((FPolₘₐₓ−FPol_{baseline})/(1+EC₅₀/[FK506]))+FPOl_{baseline}. Four-six determinations will made and presented as the mean±SEM.

### Example 14

### Fluorescence Polarization for Measuring Protein Conformational Changes

NBD-labeled hAbl is produced as in Example 9 with an unnatural amino acid comprising NBD incorporated at position 152 (i.e., in place of Ser152). The NBD-labeled unnatural amino acid undergoes a change in rotational mobility as the protein transitions from an active conformational state to an inactive conformational state. The conformational change that occurs when the kinase transitions from the inactive to the active state would result in a change in the fluorescence polarization signal of the incorporated fluorophore. Fluorescence polarization is measured as in Example 13.

### Example 15

### FRET Assay

Using the present methods, a fluorescent unnatural amino acid is incorporated at two different amino acid positions in a protein which can exist in two different conformational states. The two locations are chosen so that the two fluorescent unnatural amino acids are in sufficiently close proximity when the protein is in one of the two conformational states that a FRET interaction can take place between the two fluorescent unnatural amino acids (generally, less than 25 nanometers, more preferably within 10 nanometers). The positions of these fluorescent unnatural amino acids however changes when the protein assumes the other conformational state, resulting in a changed optical signal due to a changed FRET interaction between the fluorescent moieties of the unnatural amino acids, i.e. the signal will be of greater intensity if the fluorophores are moved closer together as a result of a conformational change and will be of lesser intensity if they are moved further apart. Since the fluorescent moieties are the same, the FRET interaction between them (a homo-FRET interaction) results in a depolarized fluorescent emission. A change in the conformational state of the protein is therefore monitored using fluorescence anisotropy (as disclosed, e.g., in U.S. Patent Publication No. 20060275822).

### Example 16

### Protein Aggregation Assay

Aggregation of amyloid precursor protein (APP) fragments (associated with Alzheimer's disease) in a mammalian cell is monitored by incorporating an unnatural amino acid labeled with NBD into APP in the cell using the present methods. NBD is incorporated into a residue in the N-terminal region (residues 1-42) of APP, where it is exposed to a relatively aqueous environment. The formation of Aβ42-NBD by the cleavage of APP-NBD results in the formation of oligomers and eventually fibrils, and the aggregation of such molecules “buries” the NBD fluorophore in a hydrophobic environment, resulting in a significant increase in the fluorescent signal. The increase in fluorescence thus serves as a measure of Aβ42 aggregation. A therapeutic compound affecting the amount or rate of aggregation of APP fragments can be screened with the foregoing assay by exposing a mammalian cell comprising the labeled APP molecule to the compound and comparing the amount or rate of aggregation in that cell with the amount or rate of aggregation in a cell not exposed to the compound.

Although the present invention has been discussed in considerable detail with reference to certain preferred embodiments, other embodiments are possible. The steps disclosed for the present methods are not intended to be limiting nor are they intended to indicate that each step depicted is essential to the method, but instead are exemplary steps only. As will be understood by those of skill in the art with reference to this disclosure, the actual dimensions of any device or part of a device disclosed herein, and the actual volumes, amounts, time periods, and other quantities recited in the process and method steps in this disclosure, will be determined by the intended use of such device or the intended application of such process or method. Therefore, the scope of the appended claims should not be limited to the description of preferred embodiments contained in this disclosure. All references cited herein are incorporated by reference to their entirety.

1. A method of performing an assay to determine a property of a protein, comprising the steps of:
providing a translation system comprising tRNAs and aminoacyl synthetases;
providing an O-tRNA/O-RS pair which is orthogonal to the tRNAs and aminoacyl synthetases of the translation system, wherein the O-tRNA is aminoacylated by the O-RS with a label, and wherein the label is an unnatural amino acid molecule comprising a fluorescent moiety or a reactive unnatural amino acid molecule;
providing an mRNA molecule coding for the protein, wherein the mRNA molecule comprises a selector codon;
translating the mRNA molecule with the translation system and the O-tRNA/O-RS pair, wherein the O-tRNA comprises an anticodon loop that specifically binds the selector codon of the mRNA molecule, thereby site-selectively incorporating the label into the protein;
if the label comprises a reactive unnatural amino acid molecule, providing a fluorescent molecule comprising a reactive moiety and a fluorescent moiety, and reacting the reactive unnatural amino acid with the reactive moiety of the fluorescent molecule, thereby attaching the fluorescent moiety to the label;
exciting the fluorescent moiety of the label; and
measuring an emitted optical signal produced in response to the excitation of the fluorescent moiety, thereby determining the property of the protein.

2. The method of claim 1, wherein the translation system comprises components of a eukaryotic cell.

3. The method of claim 1, wherein the fluorescent moiety of the label is a polarity-sensitive fluorophore.

4. The method of claim 1, wherein the fluorescent moiety of the label is a polarity-sensitive fluorophore, and wherein when the polarity-sensitive fluorophore is incorporated into the protein it is exposed to a hydrophobic environment when the protein is in a first conformational state and is exposed to a hydrophilic environment when the protein is in a second conformational state, and wherein the property of the protein determined is the conformational state of the protein.

5. The method of claim 4, further comprising the steps of contacting the protein with a target molecule and determining the conformational state of the protein in the presence of the target molecule.

6. The method of claim 1, wherein the protein is an enzyme.

7. (canceled)

8. The method of claim 6, wherein the protein is a kinase and the target molecule binds outside of the kinase's ATP-binding site.

9. (canceled)

10. The method of claim 1, wherein the cell is a yeast cell.

11-13. (canceled)

14. The method of claim 1, wherein the assay is performed in vitro.

15. The method of claim 1, wherein the assay is performed in a cell and wherein the fluorescent moiety is excited while it is in the cell.

16-17. (canceled)

18. The method of claim 1, wherein the O-RS is derived from a tyrosyl aminoacyl synthetase.

19. The method of claim 1, wherein the fluorescent moiety is excited with polarized light

20. (canceled)

21. The method of claim 1, wherein the unnatural amino acid is a derivative of a natural amino acid comprising a substitution or addition selected from the group consisting of an alkyl group, an aryl group, an acyl group, an azido group, a cyano group, a halo group, a hydrazine group, a hydrazide group, a hydroxyl group, an alkenyl group, an alkynl group, an ether group, a thiol group, a sulfonyl group, a seleno group, an ester group, a thioacid group, a borate group, a boronate group, a phospho group, a phosphono group, a phosphine group, a heterocyclic group, an enone group, an imine group, an aldehyde group, a hydroxylamino group, a keto group, a sugar group, α-hydroxy group, a cyclopropyl group, a cyclobutyl group, a cyclopentyl group, a 2-nitrobenzyl group, a 3,5-dimethoxy-2-nitrobenzyl group, a 3,5-dimethoxy-2-nitroveratrole carbamate group, a nitrobenzyl group, a 3,5-dimethoxy-2-nitrobenzyl group, and an amino group.

22. (canceled)

23. The method of claim 1, wherein the unnatural amino acid is a reactive unnatural amino acid selected from the group consisting of 2-F-phenylalanine, 3-F-phenylalanine, 4-F-phenylalanine, 2-Br-phenylalanine, 3-Br-phenylalanine, 4-Br-phenylalanine, 2-Cl-phenylalanine, 3-Cl-phenylalanine, 4-Cl-phenylalanine, 4-CN-phenylalanine, p-azido-phenylalanine, o-azido-phenylalanine, 2-amino-2-(4-(ethynyloxy)phenyl)acetic acid, p-acetyl-phenylalanine, 2-(4-allylphenyl)-2-aminoacetic acid, p-ethynyl-phenylalanine, 2-amino-4-oxopentanoic acid, and 2-amino-5-oxohexanoic acid.

24. (canceled)

25. The method of claim 1, wherein the fluorescent moiety is selected from the group consisting of a dansyl group, an anthraniloyl group, an acrylodan group, a coumarin group, a 4-nitrobenzo[c][1,2,5]oxadiazole (NBD) group, and a dipyrrometheneboron difluoride (BODIPY) group.

26. The method of claim 1, wherein the fluorescent moiety is selected from the group consisting of 4-nitrobenzo[c][1,2,5]oxadiazole (NBD), acrylodan, dansylalanine, dansylysine, dansyl-dap, 7-azatryptophan, 3-anthraniloyl-2-amino propionic acid (AtnDap), 6-dimethylamino-2-acyl-napthalene alanine, (ALADAN), α-amino-3-[6,7dimethoxy-2-oxo-2H-chromen-4-ylmethyl)-amino]-propionic acid, 2-amino-3-(7-nitro-benzo[1,2,5]oxadiazol-4-ylamino)propionic acid (NBD-Dap), 2-amino-3-BODIPY-propionic acid, 2-amino-6-BODIPY-hexanoic acid, 2-hydroxy-3-BODIY-propionic acid, and 2-hydroxy-6-BODIPY-hexanoic acid.

27. The method of claim 1, wherein the fluorescent moiety is selected from the group consisting of a dansyl group, an anthraniloyl group, an acrylodan group, a coumarin group, an 4-nitrobenzo[c][1,2,5]oxadiazole (NBD) group, a fluorescein group, and a dipyrrometheneboron difluoride (BODIPY) group, and wherein the fluorescent moiety comprises a reactive moiety selected from the group consisting of an alcohol moiety, a hydrazide moiety, an acetylene moiety, and an azide moiety, wherein the reactive moiety can be coupled to a reactive unnatural amino acid.

28. The method of claim 1, wherein the property being determined is selected from the group consisting of protein conformation, dimerization of the protein, and aggregation of the protein.

29-31. (canceled)

32. A system for performing any of the foregoing methods, comprising:
a translation system comprising tRNAs and aminoacyl synthetases;
an O-tRNA/O-RS pair which is orthogonal to the tRNAs and aminoacyl synthetases of the translation system;
a label comprising a fluorescent unnatural amino acid molecule or a reactive unnatural amino acid molecule, wherein the O-tRNA is aminoacylated by the O-RS with the fluorescent unnatural amino acid molecule or the reactive unnatural amino acid molecule;
an mRNA molecule coding for a protein, the mRNA molecule comprising a selector codon; and
a fluorescent molecule comprising a reactive moiety and a fluorescent moiety, if the label comprises a reactive unnatural amino acid molecule.

33-35. (canceled)


SEQUENCE LISTING (13 sequences)

SEQ ID NO: 1 - DNA, 81 bp, Artificial
  Modified Lactococcus lactis tyrosyl tRNA
ggaagggtagcgaagtggctaaacgcggcggactctaaatccgctccttcgggttcgggggttcgaatccctccccttcca

SEQ ID NO: 2 - DNA, 170 bp, Artificial
  Modified Lactococcus lactis tyrosyl tRNA for expression in mammalian cells
gaattcagcgctccggtttttctgtgctgaacctcaggggacgccgacacacgtacacgtcggaagggtagcgaagtggctaaacgcggcggactctaaatccgctccttcgggttcgggggttcgaatccctccccttccagacaagtgcggtttttttctccggatcc

SEQ ID NO: 3 - DNA, 170 bp, Artificial
  Modified Lactococcus lactis tyrosyl tRNA for expression in yeast
gctagcaagcttcccgataagggagcaggccagtaaaaagcattaccccgtggggaagggtagcgaagtggctaaacgcggcggactctaaatccgctccttcgggttcgggggttcgaatccctccccttccaccatttttttcaaaagtccctgaacttcccaccggt

SEQ ID NO: 4 - DNA, 113 bp, Artificial
  Modified Lactococcus lactis tyrosyl tRNA for expression in Xenopus oocytes
taatacgactcactataggaagggtagcgaagtggctaaacgcggcggactctaaatccgctccttcgggttcgggggttcgaatccctccccttccaccatgagacccatcc

SEQ ID NO: 5 - DNA, 183 bp, Artificial
  Modified E. coli leucyl tRNA gene for expression in yeast cells
gcggccgcgtcgacctctttttcaattgtatatgtgttatgtagtatactctttcttcaacaattaaatagcccggatggtggaatcggtagacacaagggattctaaatccctcggcgttcgcgctgtgcgggttcaagtcccgctccgggtatttttttgttttttatgctcgagactagt

SEQ ID NO: 6 - DNA, 185 bp, Artificial
  Modified E. coli leucyl tRNA gene for expression in mammalian cells
ctcgagactagtagcgctccggtttttctgtgctgaacctcaggggacgccgacacacgtacacgtcgcccggatggcggagtcggtagacgcaagggattctaaatccctcggcgttcgcgctgtgcgggttcgagtcccgctccgggtagacaagtgcggtttttttctcctctagatccgga

SEQ ID NO: 7 - DNA, 185 bp, Artificial
  Modified E. coli leucyl tRNA gene for expression in mammalian cells
tctagatccggaccagattaaatgcaaaagcaaaagcattaagtagctttctggcataaacatattgcagctggcccggatggcggagtcggtagacgcaagggattctaaatccctcggcgttcgcgctgtgcgggttcgagtcccgctccgggtagggttgctgtctttttgtcccccccggg

SEQ ID NO: 8 - DNA, 1260 bp, Lactococcus lactis
  CDS (1)..(1260)
atgaacatttttgacgaacttaaagcacgtggtttggttttccaaacaacggacgaagctgcgctttctaaagcgctgacggaggacatggtttcctattatgtaggatatgaccctaccgctgattcccttcatttaggtaatcttgtactaattttgacaatgaaacgtctacaaatggctggacataaaccttatgcgcttgttggtggtgcaactggtttgattggtgatccttctttcaaagattcagaacgttcacttcaaacaaaagacactgtaacaaaatggtctggaaaaatccgtagtcagcttgagcgtttcctggattttgaaaatggtgaaaataaggcagaaatgaccaataactacaactggtttgaaaatctgacttttattgatttcctcagagatgtgggtaaacatttcaccgttaactacatgatttctaaagactctgtaaaatcacggatggaaagtggaatttcttatacagaatttgcttaccaaattatgcaaggttatgatttctacgaattgaaccaattgcacaatgttactttacaacttggtggttctgatcaatggggaaatatgactgctggaacagagcttttacgtcgtaaagcgaacaagcaaggtcatgttattactattccattaattacagattcaactggtaaaaaattcggtaaatcagaaggaaatgcaatttggcttgatgctgataaaacatctccttatgaaatgtaccaattttggcttaatgttgatgatgcagatgctgtaaaaatgttgaaaattttcaccttcctttcattagaagagattgctgaaattgaagaacaatttgaagcagctcgtcatgagcgtttggcccaaaaagttttggctcgtgaagttgtcagcttagttcacggaaaagcagcttatgaacaagcagttaaaacatcagaaatcttatttggcggcggagatttgcgtcaattagatgctaaatcaatcttaactggtcttaaagcagctccacaacatcaagttactgatgatgaagatttaactttagttgaacttttaatctctgctggaatagctccttcaaaaagacaagctcgtgaagatattacaaatggtgcgatttatatcaatggtgaacgtgttcaagcacttgattatgttattacagattcagataaaattgaaaatcgtttaactgtcattcgtcgtgggaaaaagaaaaacttcgttttgacttattaa

SEQ ID NO: 9 - protein, 419 aa, Lactococcus lactis
MNIFDELKARGLVFQTTDEAALSKALTEDMVSYYVGYDPTADSLHLGNLVLILTMKRLQMAGHKPYALVGGATGLIGDPSFKDSERSLQTKDTVTKWSGKIRSQLERFLDFENGENKAEMTNNYNWFENLTFIDFLRDVGKHFTVNYMISKDSVKSRMESGISYTEFAYQIMQGYDFYELNQLHNVTLQLGGSDQWGNMTAGTELLRRKANKQGHVITIPLITDSTGKKFGKSEGNAIWLDADKTSPYEMYQFWLNVDDADAVKMLKIFTFLSLEEIAEIEEQFEAARHERLAQKVLAREVVSLVHGKAAYEQAVKTSEILFGGGDLRQLDAKSILTGLKAAPQHQVTDDEDLTLVELLISAGIAPSKRQAREDITNGAIYINGERVQALDYVITDSDKIENRLTVIRRGKKKNFVLTY

SEQ ID NO: 10 - DNA, 1329 bp, Artificial
  Humanized Lactococcus lactis wildtype tyrosyl synthetase mutant
atgaatatcttcgatgaactcaaagcaaggggcctggtgtttcaaacaaccgatgaggccgcactttccaaagccctgacagaagatatggtgagctactacgtggggtacgatcccactgcagatagtctgcacctcggaaatctggttttgatactgacaatgaaaaggttgcagatggcagggcacaagccctacgccctggtcggaggcgccactggcttgattggcgatccctcattcaaagattcagagcggtccttgcagacaaaggacacggtgaccaagtggagtggaaaaatacgatcccagctggagaggtttttggatttcgagaatggcgagaacaaagccgagatgactaacaattacaactggtttgagaacctcacctttattgacttcctgcgggacgttggcaaacactttacggttaactatatgatttcaaaggattccgtgaagtcccggatggagtctggaattagctataccgagttcgcttaccaaatcatgcagggatacgatttctacgagctcaatcaacttcataatgtgacactgcagctcggcgggagcgaccagtggggcaacatgaccgctggaacagagctcctgcgccggaaggcaaataagcagggtcacgttattaccattccacttatcacagactccaccggaaagaaattcgggaaatccgagggaaacgctatttggctcgatgccgataaaacatctccatacgaaatgtatcagttctggctgaatgtagatgacgccgatgcagtgaagatgcttaaaatctttacattcctgagcctggaagagatcgctgaaatagaggaacagtttgaggccgctagacatgagcgcctggcgcagaaggttctcgcccgagaggtggtgtcactggttcacgggaaggccgcatacgaacaggcagtcaaaacatctgaaatactcttcggagggggtgacctcaggcaactggacgccaagtctatcttgaccgggctcaaagccgctccacagcaccaagtgactgatgatgaggatctgacactcgtggagcttttgatctctgccggcatagctccatccaaacggcaggcaagggaggatattacaaacggtgccatctacatcaacggcgaacgcgtacaggcacttgactatgtgatcactgacagcgacaaaatagaaaacagactgacggtcatcaggcggggtaaaaagaagaatttcgtcctgacgtatggtaagcccataccgaatcctttgctgggactcgacagcacaaggactggtcaccaccaccatcaccactaa

SEQ ID NO: 11 - protein, 442 aa, Artificial
  Synthetic Construct
MNIFDELKARGLVFQTTDEAALSKALTEDMVSYYVGYDPTADSLHLGNLVLILTMKRLQMAGHKPYALVGGATGLIGDPSFKDSERSLQTKDTVTKWSGKIRSQLERFLDFENGENKAEMTNNYNWFENLTFIDFLRDVGKHFTVNYMISKDSVKSRMESGISYTEFAYQIMQGYDFYELNQLHNVTLQLGGSDQWGNMTAGTELLRRKANKQGHVITIPLITDSTGKKFGKSEGNAIWLDADKTSPYEMYQFWLNVDDADAVKMLKIFTFLSLEEIAEIEEQFEAARHERLAQKVLAREVVSLVHGKAAYEQAVKTSEILFGGGDLRQLDAKSILTGLKAAPQHQVTDDEDLTLVELLISAGIAPSKRQAREDITNGAIYINGERVQALDYVITDSDKIENRLTVIRRGKKKNFVLTYGKPIPNPLLGLDSTRTGHHHHHH

SEQ ID NO: 12 - DNA, 2601 bp, Artificial
  E. coli “Humanized” leucyl synthetase mutant for incorporation of dansyl-alanine
atgcaggaacagtataggcctgaagaaatcgaatccaaagtccagttgcattgggatgagaaacggacattcgaagtcactgaagacgagtcaaaagaaaagtactattgcctctctgctaacccttatccctcaggcagactgcacatgggacatgtcagaaattacactattggcgatgtaatcgccagatatcagaggatgcttgggaagaatgtgttgcagccgattggatgggacgcctttgggcttcctgccgagggtgccgccgtgaagaacaacacagcaccagctccatggacttacgataatatcgcctacatgaagaatcagttgaagatgctcggattcggatatgattggagcagggagctggctacctgtacacccgagtattataggtgggagcagaaattctttactgaattgtacaagaaaggcttggtctacaagaagacgagtgccgttaactggtgcccaaatgaccaaaccgtgcttgctaatgaacaggtgattgacggctgttgctggcggtgcgacaccaaagtggaaaggaaagagattcctcagtggtttattaagatcaccgcctatgcggacgagctgctcaatgacctggataaactcgatcactggccggacactgttaagactatgcagcggaattggatcggaagaagtgagggcgtcgagataacatttaatgtgaatgactatgacaacactctgacagtgtatacgacccggcctgacgcctttatgggctgcacgtatctcgctgtggctgcaggacacccgctggcccagaaggcagctgagaacaaccccgaactggccgcattcattgatgagtgcaggaatacgaaggttgcagaggcggagatggcaaccatggagaagaagggagtggacaccggcttcaaggcggtgcaccctttgacaggcgaggaaattccagtgtgggcagctaactttgtgctcatggagtacggcacgggggccgttatggcagtcccagggcacgatcagagggattatgagtttgccagcaagtatggattgaacataaaaccagtgatactcgctgccgacggctccgagccagatctcagtcagcaggctcttaccgaaaagggggtgttgtttaactccggggagttcaatggcttggaccacgaagcagctttcaatgctatcgccgataaactcacagcgatgggtgtcggggagaggaaggtgaattatcgcctccgggattggggtgtgagtaggcagcggtattggggtgcccccatcccaatggtaacacttgaggacgggaccgttatgccaacccccgatgatcaactccccgttattctgcccgaggacgtggtgatggacggaatcacgtcccctatcaaagcagaccccgagtgggcgaagacgacggttaatggcatgcccgcactgcgggagacagacaccttcgacacattcatggagtcctgttggatatatgccagatatacctgccctcagtataaggaaggcatgctggactccgaggctgccaactactggttgccggtggatattgggataggcgggattgaacacgccatcatgaccctcctgtacttcaggtttttccacaaactgatgagagacgctggtatggttaatagcgatgaacctgctaagcagttgctctgccagggtatggtgctggcggacgctttttactacgttggggaaaatggagagaggaattgggtgagccccgtggacgccatcgtagaacgggacgaaaagggtaggattgtgaaggccaaggatgccgccggccacgagctggtctataccggaatgagcaaaatgtctaaatctaagaataatggtatcgacccacaagtgatggtggaacgatacggcgctgatacggtcagactctttatgatgttcgctagccctgcggatatgacactcgagtggcaggagtcaggcgtggaaggcgcaaaccgcttccttaaacgggtctggaaactggtgtacgagcatacagccaagggtgatgtggccgcacttaacgtggacgccttgactgaaaaccaaaaagcgcttaggagggacgtacacaaaacaatcgccaaagtgacagacgatataggaaggcgccagacctttaataccgcgatcgccgccattatggagcttatgaataagctcgccaaggctccgaccgatggtgagcaggaccgcgcactcatgcaggaagcactgctggcggttgtcagaatgctcaatcccttcactccacacatctgtttcaccctttggcaggagctcaagggggagggggatatcgacaatgctccctggcctgtcgctgacgagaaggccatggtcgaggattcaacactggtcgtcgtgcaggtcaacggcaaagttagggccaaaatcaccgtacctgtggatgctaccgaggaacaggtgcgcgaacgagccggccaggagcacttggtggccaaatatctggacggggttactgtcagaaaggttatttacgtaccagggaaactcctgaacctggtcgtgggacaccatcaccaccaccactaa

SEQ ID NO: 13 - protein, 866 aa, Artificial
  Synthetic Construct
MQEQYRPEEIESKVQLHWDEKRTFEVTEDESKEKYYCLSANPYPSGRLHMGHVRNYTIGDVIARYQRMLGKNVLQPIGWDAFGLPAEGAAVKNNTAPAPWTYDNIAYMKNQLKMLGFGYDWSRELATCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDAFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWGAPIPMVTLEDGTVMPTPDDQLPVILPEDVVMDGITSPIKADPEWAKTTVNGMPALRETDTFDTFMESCWIYARYTCPQYKEGMLDSEAANYWLPVDIGIGGIEHAIMTLLYFRFFHKLMRDAGMVNSDEPAKQLLCQGMVLADAFYYVGENGERNWVSPVDAIVERDEKGRIVKAKDAAGHELVYTGMSKMSKSKNNGIDPQVMVERYGADTVRLFMMFASPADMTLEWQESGVEGANRFLKRVWKLVYEHTAKGDVAALNVDALTENQKALRRDVHKTIAKVTDDIGRRQTFNTAIAAIMELMNKLAKAPTDGEQDRALMQEALLAVVRMLNPFTPHICFTLWQELKGEGDIDNAPWPVADEKAMVEDSTLVVVQVNGKVRAKITVPVDATEEQVRERAGQEHLVAKYLDGVTVRKVIYVPGKLLNLVVGHHHHHH